(19) United States
(12) Patent Application Publication
(10) Pub. No.: US 2002/0037514 A1
(43) Pub. Date: Mar. 28, 2002

(54) Identification of nuclear receptor-dependent coregulator recruitment

(76) Inventors: Elliott S. Klein, Danbury, CT (US); Weizhen Wang, Newport Coast, CA (US); Roshantha A. Chandraratna, Laguna Hills, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 09/815,156

(22) Filed: Mar. 22, 2001

Related U.S. Application Data
(60) Provisional application No. 60/192,036, filed on Mar. 24, 2000.

(51) Int. Cl. C12Q 1/68

(52) U.S. Cl. 435/6

(57) ABSTRACT

Methods for identifying molecules that modulate nuclear receptor transactivation activity. Also disclosed are methods for detecting endogenous nuclear receptor co-factors with increased sensitivity.

This application is based on, and claims the benefit of, U.S. Provisional Application No. 60/192,036, filed Mar. 24, 2000, and entitled “IDENTIFICATION OF NUCLEAR RECEPTOR-DEPENDENT COREGULATOR RECRUITMENT,” and which is incorporated herein by reference.

### BACKGROUND OF THE INVENTION

A vast array of specific metabolic, developmental, and catabolic processes appear to be directly or indirectly regulated in vivo by comparatively small molecules such as steroids, retinoids, vitamins, and thyroid hormones. The mechanism whereby a single such compound can contribute to the regulation of numerous different cellular events was the subject of much speculation until relatively recently, when it was discovered that these compounds each share the ability to bind to transcriptionally active proteinaceous receptors. These protein receptors, in turn, are able to bind specific cis-acting nucleic acid regulatory sequence regions, termed response elements or RE's, located upstream of the coding sequence of certain genes and to activate the transcription of these genes. Thus, the proteinaceous receptors can serve as specific, ligand-dependent regulators of gene transcription and expression.

The amino acid sequences of these various receptors were quickly found to share regions of relative homology, thus making each such receptor a member of a family of ligand-modulated receptor molecules. This family has been termed the steroid superfamily of nuclear hormone receptors; nuclear, because the receptors are usually found in high concentration in the nucleus of the cell.

Further study of the structural and functional relationship between the nuclear hormone receptors has shown certain characteristics in common between them in addition to sequence homology. (See e.g., Evans et al. Science 240:889-895 (1988); this and all other references cited in this application are hereby incorporated by reference as part of this specification unless expressly indicated otherwise.) For example, the known nuclear receptors bind response elements upstream of their regulated genes in the form of a dimer.

One class of the nuclear hormone receptors, including the glucocorticoid, estrogen, androgen, progestin, and mineralcorticoid receptors have been found to bind as homodimers to specific response elements organized as inverted repeats. Another class of nuclear hormone receptors, which includes the retinoid receptor RAR (retinoic acid receptor), the thyroid receptor(TR), the vitamin D receptor (VDR), the farnesoid X receptor (FXR), oxysterol receptor (LXR), the peroxisome proliferator receptor (PPAR), and the insect ecdysone receptor bind their response elements as a heterodimer in conjunction with the retinoid X receptor (RXR), which in turn is positively activated by 9-cis retinoic acid. See Mangelsdorf, et al., The Retinoid Receptors in The Retinoids: Biology, Chemistry and Medicine Ch.8 (Sporn et al., eds. 2d ed., Raven Press Ltd. 1994); Nagpal and Chandraratna, Current Pharm. Design 2:295-316 (1996), which are both incorporated by reference herein.

RXR can form homodimers which are responsive to RXR-activating compounds. However, in several of the RXR-containing heterodimeric pairs, the RXR subunit appears to be a silent partner; for example, synthetic RXR agonists do not activate the RAR/RXR heterodimer. This apparent inability has been proposed to be due to allosteric inhibition of the RXR moiety in the heterodimer.

There are a sizable number of different nucleic acid response elements present in the promoters of nuclear receptor-responsive genes; these REs can be divided into groups based on functional or structural similarities or both. Taking the retinoid receptors RAR and RXR as an example, like many nuclear receptors, these receptors exist as a number of subtypes (RARα, RARβ, RARγ, and RXRα, RXRβ, and RXRγ), each encoded by a separate gene. Each subtype may exist in different isoforms. Response elements binding RAR (the RAREs) have been divided into three classes: Class I RAREs are arranged as direct hexanucleotide repeats separated by 5 random nucleotides, and are called DR-5 elements. These repeats may or may not be literal; for example in the promoter of the gene encoding the mouse RARβ2 receptor, the hexanucleotide GGTTCA is separated by 5 nucleotides from the sequence AGTTCA. These sequences are considered to be direct repeats. Here and elsewhere in this specification nucleotide sequences are written in the direction from 5′ to 3′, and amino acid sequences are written in the direction from amino to carboxyl terminus unless otherwise indicated or clear from the context to be otherwise.

Class II RAREs are those response elements having direct repeats separated by two nucleotides, and are termed DR-2 response elements.

Class III RAREs are those response elements that are neither DR-5 or DR-2 REs, and which generally have a more complex structure. Some are inverted repeats, some are separated by no nucleotides or by as many as 14 nucleotides, and some are repeated three times rather than twice.

By contrast, response elements recognized by RXR (RXREs) exist mainly as direct repeats spaced by a single nucleotide. In most cases these repeats are present in pairs, but may occur as many as five times in series, as in the RXRE for rat CRBP II.

Each unit of a RE pair is termed a “half site”, reflecting that most such RE sites comprise a pair of direct repeats, inverted repeats, or palindromes. Similar patterns are seen in the organization of response elements selective for nuclear receptors other than the retinoid receptor.

The structures of the members of the nuclear receptor superfamily are modular in nature. These regions of similarity include, from amino terminus to carboxyl terminus, regions termed A through F. Regions A and B together, located at the N-terminus of the receptor, comprise a transactivation function known as AF-1. In the retinoid receptor RAR, the A/B domain modulates transcription independently of receptor ligand binding. Region C is a highly conserved domain that functions as the DNA binding domain (DBD) and is responsive to cognate cis-acting response elements. The presence in this region of two cysteine-rich zinc fingers, common among other DNA binding proteins, facilitates critical interactions with specific nucleotide sequences of the RAREs. Next to the DNA binding domain is region D, or the hinge domain. Region E contains a ligand binding domain (LBD), which serves a retinoid-dependent activation function, referred to as AF-2, and a dimerization function, which promotes the association of receptor molecules as dimers. This latter region contains hydrophobic leucine zipper motifs. The function of region F, located at the C-terminus, is still largely unknown. RXRs, and perhaps other nuclear receptors, lack region F.

The retinoid receptors have been implicated as regulators of cell growth, differentiation, metabolism, hematopoiesis, and bone development. Additionally, there is evidence that retinoids may have antiproliferative activity, and therefore may be useful in the treatment of cancer. The other nuclear hormone receptors are also regulators of gene expression, and therefore also play an important part in development, maturation, and adaptability of the organism to its environment.

As indicated above, the regulation of gene transcription by nuclear hormone receptors is a ligand-dependent phenomenon. Although not wishing to be limited by theory, the binding of a receptor ligand to its cognate receptor is believed to result in a conformational change whose result is either a positive or negative regulation of gene transcription. However, the mechanism by which transcription is controlled has recently been the subject of much intense research.

As a result of this research it has become clear in the last few years that molecules other than the ligand and the nuclear receptor molecules themselves are also involved in regulation of hormone receptor-mediated gene transcription. For example, it was discovered that a certain class of retinoic acid receptor (RAR) ligands which are able to block receptor agonist activity (by “agonist” is meant a nuclear receptor ligand that promotes the receptor-mediated stimulation of gene transcription) are also able to decrease a basal level of gene expression that normally exists in the absence of receptor ligand. Thus, rather than acting as a “neutral antagonist” or competitive inhibitor of receptor-mediated gene transcription, these compounds, subsequently termed “negative hormones” and “inverse agonists”, are actually responsible for down-regulating RAR-mediated gene transcription. See e.g., Klein et al., U.S. Pat. No. 5,776,699, hereby incorporated by reference herein.

Inverse agonists are functionally distinguishable from the neutral antagonist ligands that block the up-regulation of the receptor without evidencing a receptor stimulatory effect. These findings implied the existence of a “co-repressor” of receptor activity which is able to be recruited by the inverse agonist-bound RAR, in turn causing a depression of transcriptional activity. One model seeking to incorporate these findings postulates that binding of the inverse agonist causes a conformational change in the RAR receptor, resulting in a greater avidity of the receptor for the co-repressor. See id.

Similar findings were made in a thyroid hormone receptor (T3R) system. T3R forms a heterodimer with RXR to exert both positive and negative control of gene transcription. Repression of gene transcription by T3R occurs in the absence of ligand, and appears to involve the ligand binding domain of the T3R molecule. Horlein et al. Nature 377:397 (1995) (hereby incorporated by reference herein) have shown, using recombinant receptor constructs, the unliganded T3R/RXR heterodimer was able to bind a 270 KDa protein from a cell free extract of CV-1 cells. This protein, termed p270 or N-COR (for nuclear receptor co-repressor), was demonstrated to bind the heterodimer only in the absence of thyroid hormone, while the addition of a receptor agonist prevented binding between N-COR and the receptor under the tested experimental conditions.

The N-CoR protein also appears to bind RAR in the absence of ligand. In both cases N-CoR binds the hinge domain of the nuclear receptors, located between the DBD and the LBD of the receptor proteins. Experiments using the cloned N-COR protein demonstrated that N-CoR mediates a 15 to 25-fold repression of transcription on T3R/RXR and RAR/RXR heterodimers when the heterodimers are bound to their respective DNA response elements. See Horlein, id.

Another co-repressor termed SMRT has been characterized. Chen et al., Nature 377:454 (1995), hereby incorporated by reference herein. This co-repressor was first characterized as binding RAR and the thyroid hormone receptor (TR) in the absence of ligand. In both cases, this protein disassociates from the receptors upon the incubation of the receptor with a ligand agonist.

Recent work has demonstrated that both SMRT and N-COR are able to regulate transcription activation, not only by RAR and TR, but also by PPARγ, the estrogen receptor (ER), and the progesterone receptor (PR). Lavinsky et al., Proc. Natl. Acad. Sci. 95:2920 (1998), incorporated by reference herein. Additionally, it is to be expected that there are other receptors mediated by SMRT and N-COR as well as other co-repressors acting similarly whose activities have not been as well characterized.

In addition to nuclear receptor co-repressors, a family of nuclear receptor co-activators have also been characterized. In the case of RAR/RXR heterodimers, Kurokawa et al., Nature 377:451-454 (Oct. 5, 1995), (incorporated by reference herein) demonstrated that two PAGE bands having apparent molecular weights of 140 KDa and 160 KDa contain proteins that bind to the RAR component of the heterodimer in the presence of the RAR specific agonist TTNTB or the RAR and RXR agonist 9 cis retinoic acid (9-cis RA). This publication is hereby incorporated by reference herein. The addition of an RXR-specific agonist did not induce an interaction between the heterodimer and the p140 or p160 protein species.

Several lines of evidence suggested that the p140 and p160 bands contain co-activators of heterodimer-mediated transcription. The interaction of these protein species required the AF-2 domain of the RAR DBD; this domain is conserved among many members of the nuclear receptor superfamily, and is essential for ligand-dependent transcription. Also, the p140 and p160 proteins were unable to bind RAR in the presence of RAR antagonists which prevent the binding of an RAR agonist to the RAR LBD. While not wishing to be limited by theory, these results suggest that ligand binding results in a conformational shift in the RAR molecule that is required for both p140/p160 binding and for transcriptional activation.

Interestingly, the RAR/RXR heterodimer binds a DR-5 response element with the RAR portion of the heterodimer bound to the 3′ DR-5 half site, but binds a DR-1 RE with the RAR portion of the heterodimer at the 5′ half site. See Kurokawa et al., supra. This DNA-induced difference in polarity does not appear to affect the binding of the p140 or p160 protein binding. However, the addition of an RAR agonist to the DR-1-bound heterodimer does not result in transcriptional activation or the dissociation of N-CoR from the complex.

Several discrete nuclear receptor co-activators have now been identified and the nucleotide sequences of the mRNA encoding them have been determined. Among such co-activators are human SRC-1, TIF2, ACTR and N-COA; it is likely that the p160 band referred to above contained SCR-1 and/or ACTR. While the binding characteristics of these molecules have not been thoroughly determined, these are known to bind RAR and TR. These molecules share the agonist-dependant binding characteristics described above and are necessary for transcriptional activation. It is likely that other co-activators and co-repressors of members of the steroid hormone nuclear receptor superfamily will be characterized in the near future.

Other molecules, possibly accessories or chaperones for the receptor-bound co-activators and/or co-repressors, are also thought to be respectively necessary for transcriptional activation or repression. For example CBP interacts with co-activators and sin3A interacts with co-repressors. All molecules that form a complex with nuclear receptors in a ligand dependent manner are intended to be within the definition of nuclear receptor co-factor.

### SUMMARY OF THE INVENTION

The present invention is directed to methods of detecting and measuring the interaction of co-activators, co-repressors and other accessory molecules able to directly or indirectly associate with members of the nuclear hormone receptor superfamily in a ligand-dependent manner. Thus, in a preferred embodiment, the methods are useful as methods for screening potential receptor ligands that influence the association of such receptor co-repressors, co-activators and other co-factors involved in the regulation of nuclear receptor activity.

Thus, in one embodiment, the invention involves the addition to the assay mix of a nucleic acid template having a nucleotide sequence comprising a nuclear receptor response element. It will be understood that such nuclear receptor response elements typically contain two short “half site” nucleotide sequences separated by one or more variable nucleotide. In the present assay methods, the added nucleic acid may comprise a naturally occurring nuclear receptor response element such as the RAR DR-5 response element (hereinafter termed a “RARE”) AGGTCANNNNNAGGTCA (SEQ ID NO: 1) containing two RAR selective half sites; may comprise a hybrid response element (RE) containing half sites specific for different nuclear receptors, such as, without limitation, one half site specific for RAR and another half site specific for the thyroid hormone receptor (TR). One or more half sites may be employed, although the number of half sites will normally be two. Additionally, the number of nucleotides separating such half sites may be varied as the user wishes. Such variation of “spacer” nucleotides will normally be expected to affect the conformation of the dimer as it sits on the nucleic acid template. As used in the claims, unless otherwise indicated, the term “response element” or “RE” shall mean any of the above. Unless otherwise indicated, nucleotide sequences disclosed herein are written (from left to right) in the direction 5′ to 3′, and amino acid sequences in the direction amino to carboxyl terminus.

In its most basic form, a preferred embodiment of the invention comprises a method of determining whether a compound modulates the transcriptional activity of a nuclear receptor dimer comprising the steps:

1) contacting a first nuclear receptor subunit and an optional second nuclear receptor subunit different from said first nuclear receptor subunit, with;

a) a nucleic acid comprising a response element able to bind both subunits of a nuclear receptor homo- or heterodimer comprising said first nuclear receptor subunit and said second nuclear receptor subunit, if present;

b) a compound comprising a prospective ligand of said first nuclear receptor subunit; and

c) a nuclear receptor co-factor capable of directly or indirectly binding either said first nuclear receptor subunit, or said second nuclear receptor subunit, if present, in a ligand dependent manner; and

2) detecting the association or dissociation of said co-factor with said first or second nuclear receptor subunit in the presence of said compound when compared to performing step 1) in the absence of said compound, as an indication that said compound modulates the transcription regulatory activity of said first or second nuclear receptor subunit.

The method is particularly useful in detecting the association (or dissociation) of indigenous, full length co-activators, co-repressors and other co-factors in complexes with nuclear receptors. Unlike other methods which rely on over-expression of such nuclear receptor co-factors by means of introduction of an expression or integration plasmid into a host cell, in preferred embodiments the present invention, due to its sensitivity, has the advantage of using the naturally occurring co-repressors, co-activators and accessory molecules in the intracellular amounts in which they are naturally present. In these embodiments, the assay system more closely mimics naturally-occurring transcriptional regulation by nuclear receptors than is the case when co-modulators are present in excess or as fusion proteins having heterologous amino acid sequences.

### BRIEF DESCRIPTION OF THE DRAWINGS

FIG. 1A is a plot showing the transactivation profile of selected RAR/RXR heterodimers upon incubation of CV-1 cells with various concentrations of all-trans retinoid acid (ATRA).

FIG. 1B is a plot showing the transactivation profile of selected RAR/RXR heterodimers upon incubation of CV-1 cells with various concentrations of AGN 192620.

FIG. 1C is a plot showing the transactivation profile of selected RAR/RXR heterodimers upon incubation of CV-1 cells with various concentrations of AGN 194204.

FIG. 2 is a Western blot of protein from cell lysates of CV-1 cells co-transfected with vectors expressing RARβ-VS and RXRα in the presence or absence of the RAR agonist TTNPB, the RXR agonist 194204, and a double-stranded DR-5 RARE. Proteins were immunoprecipitated with an antibody against the V5 epitope. Electrophoretically separated immunoprecipitates were detected using an anti SRC-1 antibody.

FIG. 3 is a Western blot of protein from cell lysates of CV-1 cells co-transfected with vectors expressing RARβ-VS and RXRα in the presence or absence of the RAR agonist TTNPB, a double-stranded DR-5 RARE and/or two glucocorticoid receptor response element half sites separated by 5 nucleotides (G-5-G). Proteins were immunoprecipitated with an antibody against the VS epitope. Electrophoretically separated immunoprecipitates were detected using an anti SRC-1 antibody and an anti RXR antibody.

FIG. 4 is a Western blot of protein from cell lysates of CV-1 cells co-transfected with vectors expressing RARα-V5 and RXRγ in the presence or absence of the RAR agonist TTNPB, the RXR agonist AGN 194204 and the RXR agonist AGN 192620, and in the presence of a double-stranded DR-5 RARE. Proteins were immunoprecipitated with an antibody against the VS epitope. Electrophoretically separated immunoprecipitates were detected using an anti SRC-1 antibody.

FIG. 5 is a Western blot of protein from cell lysates of CV-1 cells co-transfected with vectors expressing RARβ-V5 alone or in combination with either RXRα or RXRαΔC, a mutant RXR lacking helix 12. Lysates were treated with the RAR agonist TTNPB, the RXR agonist AGN 194204 or DMSO (vehicle alone) in the presence of a double-stranded DR-5 RARE. Proteins were immunoprecipitated with an antibody against the VS epitope. Electrophoretically separated immunoprecipitates were detected using an anti SRC-1 antibody (upper panel) or an anti RXRα antibody(lower panel).

FIG. 6 is a Western blot of protein from cell lysates of CV-1 cells co-transfected with vectors expressing RARγ-V5 and RXRα in the presence or absence of the RAR agonist TTNPB, the RXR inverse agonist AGN 193109, or the RXR antagonist AGN 193840, and in the presence or absence of a double-stranded DR-5 RARE. Proteins were immunoprecipitated with an antibody against the V5 epitope. Electrophoretically separated immunoprecipitates were detected using an anti N-COR antibody (upper panel) or an anti RXRα antibody (lower panel).

FIG. 7A is a Western blot of protein from cell lysates of CV-1 cells co-transfected with vectors expressing RARα-V5 and RXRα in the presence or absence of the RAR agonist TTNPB, the RXR inverse agonist AGN 193109, or the RXR antagonist AGN 193840, and in the presence of a double-stranded DR-5 RARE. Proteins were immunoprecipitated with an antibody against the V5 epitope. Electrophoretically separated immunoprecipitates were detected using an anti N-COR antibody (upper panel) or an anti RXRα antibody (lower panel).

FIG. 7B is a Western blot of protein from cell lysates of CV-1 cells co-transfected with vectors expressing RARβ-V5 and RXRα in the presence or absence of the RAR agonist TTNPB, the RXR inverse agonist AGN 193109, or the RXR antagonist AGN 193840, and in the presence of a double-stranded DR-5 RARE. Proteins were immunoprecipitated with an antibody against the V5 epitope. Electrophoretically separated immunoprecipitates were detected using an anti N-CoR antibody (upper panel) or an anti RXRα antibody (lower panel).

FIG. 8A is a Western blot of protein from cell lysates of CV-1 cells co-transfected with vectors expressing RARα-V5 and RXRY in the presence or absence of the RAR agonist all trans retinoic acid (ATRA) and a double-stranded DR-5 RARE. Lysates were also incubated with synthetic peptides comprising the LXD1, LXD2, or LXD3 amino acid sequence (see text) prior to immunoprecipitation and Western analysis. Proteins were immunoprecipitated with an antibody against the V5 epitope. Electrophoretically separated immunoprecipitates were detected using an anti SRC-1 antibody (upper panel) or an anti ACTR antibody (lower panel).

FIG. 8B is a Western blot of protein from cell lysates of CV-1 cells co-transfected with vectors expressing RARγ-V5 and RXRα in the presence of the RAR agonist ATRA and a double-stranded DR-5 RARE. Lysates were also incubated with synthetic peptides comprising the LXD1, LXD2, or LXD3 amino acid sequence (see text) prior to immunoprecipitation and Western analysis. Proteins were immunoprecipitated with an antibody against the V5 epitope. Electrophoretically separated immunoprecipitates were detected using an anti SRC-1 antibody (upper panel) or an anti ACTR antibody (lower panel).

FIG. 9 shows coactivator selective recruitment to human RARα. RARα containing ternary complexes were immunoprecipitated in the presence of the indicated ligands (1 μM). Anti-SRC-1, N-CoR, ACTR, p300 or RXRU antibodies were used to detect co-immunoprecipitated proteins as indicated.

### DETAILED DESCRIPTION OF THE INVENTION

Definitions:

Unless expressly indicated otherwise, the following words shall have the indicated meanings:

By “ligand” is meant a molecule able to preferentially (though not necessarily exclusively) bind a nuclear receptor under physiological conditions and thereby affect the nuclear receptor's ability to activate transcription of a gene controlled by a cognate response element. Included within the definition of “ligand” is an agonist, an antagonist, or an inverse agonist of that receptor.

By “agonist” is meant a nuclear receptor ligand which stimulates the transcriptional activation activity of the nuclear receptor for a gene having a response element located upstream of the translational start codon.

By “antagonist” is meant a nuclear receptor ligand that is able to bind the nuclear receptor, thereby blocking the ability of an agonist or inverse agonist of that receptor to stimulate or repress, respectively, the transcriptional activation activity of the nuclear receptor for a gene having a response element located upstream of the translational start codon.

By “nuclear receptor co-factor” is meant a co-activator, co-repressor, chaparone molecule, or other accessory molecule capable of directly or indirectly binding to or dissociating from a nuclear receptor in a ligand-dependent manner.

By “nucleic acid” is meant a polymer comprising a linear arrangement of naturally occurring or synthetic nucleotides joined by phosphodiester linkages. The most common nucleotides are adenosine 5′ phosphate, thymidine 5′ phosphate, cytidine 5′ phosphate, uracil 5′ phosphate and guanisine 5′ phosphate, but other rare nucleotides may include hypoxanthine, xanthine, methylated or methoxylated derivatives of the common nucleotides, and the like. Such nucleotides may be ribonucleotides or deoxyribonucleotides, and the nucleic acids may be RNA, DNA or hybrids thereof. Additionally, and specifically, synthetic nucleic acids such as, without limitation, peptide nucleic acids (PNAs) and those containing 2′O-methylribonucleotides derivatives are contemplated as being encompassed within the term “nucleic acid”. If the context indicates that a nucleic acid expresses a given protein, it will be understood that the referenced nucleic acid is capable of transcription or translation, and thus can comprise naturally occurring nucleotides.

By a co-factor “binding in a ligand-dependent manner” is meant that a co-factor associates with a nuclear receptor in a direct relationship, or in an inverse relationship, to the presence of a nuclear receptor ligand in a dose-respondent way.

By “nuclear receptor” is meant a member of the superfamily of protein transcription factors comprising the steroid superfamily of nuclear hormone receptors, which includes, without limitation, the retinoic acid receptor (RAR), the retinoid X receptor (RXR), the peroxisome proliferator receptor (PPAR), thyroid hormone receptor (TR), the estrogen receptor (ER), and progesterone receptor (PR). While active nuclear receptors exist as dimers, usually one subunit of the dimer will dominate the other such that a transcriptional response characteristic of that subunit will be detected in a co-transfection assay.

By “nuclear receptor subunit” is meant a nuclear receptor monomer or a chimeric protein or derivative thereof comprising the DNA-binding and ligand-binding properties of a nuclear receptor monomer.

The present invention is drawn to methods of determining whether a prospective nuclear receptor ligand modulates the transcriptional activity of a nuclear receptor dimer. Such methods comprise contacting the nuclear receptor with a nucleic acid response element while the nuclear receptor is exposed to the receptor ligand. The ability of the ligand to influence the transcription activation activity of the nuclear receptor dimer is determined by detecting the ligand-dependant association or dissociation of one or more nuclear receptor co-factor.

The ability of a nuclear receptor to stimulate transcription of a gene controlled by a cognate response element has been exploited for some time in “cotransfection” assays such as those described in Evans et al., U.S. Pat. Nos. 5,071,773 and 5,298,429, hereby incorporated by reference herein. In the assay described in these patents, two chimeric DNA molecules are created for transfection within a eukaryotic cell. One chimeric DNA construct contains a nuclear receptor DNA response element located in the promoter upstream of a gene whose expression can be easily detected. Examples of such genes are those encoding the enzymes firefly luciferase and B-galactosidase, whose activity can be detected and measured using an appropriate colorometric or fluorescent substrate. This construct is called the “reporter plasmid”. The other chimeric construct encodes a protein containing the nuclear receptor DNA-binding domain (DBD) that will bind to the response element of the first chimeric construct. The fusion protein also contains the transactivating portion and the ligand binding domain (LBD) of the nuclear receptor for which a ligand is sought to be found. This construct is called the “expression plasmid”. Upon co-transfection of both chimeric constructs into the same cell and expression of the fusion protein, the DNA-binding domain of this protein will bind the response element of the reporter plasmid. If the cell is treated with a ligand able to bind to the LBD of the chimeric protein and activate nuclear receptor mediated transactivation, the reporter gene will be expressed and is detected. See Evans, id. For example, if the LBD is an RXR LBD, then the ligand that stimulates transactivation is an RXR agonist; likewise, if the expression plasmid contains the estrogen receptor LBD, the a compound that stimulates transactivation will be an estrogen receptor agonist. Assays such as the co-transfection assay are useful for detecting the overall transcriptional effect of a given prospective nuclear receptor ligand or combination thereof.

It is now known that nuclear receptor-mediated transcriptional control requires the participation of a number of different molecules, including ligand, co-activators, co-repressors and various chaperone and accessory molecules, such as heat shock proteins and the like. Recent research, including analysis of the three dimensional structures of the “unliganded” RXRα LBD and the “liganded” RARγ LBD, has suggested that the nuclear receptors undergo an allosteric conformational change upon being bound by ligand. Also, it was found that various receptors could interfere with the transcriptional activity of each other, and that transcriptional repression by an unliganded nuclear receptor (thyroid hormone receptor) could be reversed by the addition of other nuclear receptors such as RAR and v-erbA. See e.g., Piedrafita and Pfahl, Nuclear Retinoid Receptors and Mechanisms of Action in Retinoids: The Biochemical and Molecular Basis of Vitamin A and Retinoid Action Ch. 5 p. 153-184 (ed. Heinz Nau & William S. Blaner 1999), incorporated by reference herein. These results suggested the existence of limiting amounts of molecules having “co-activator” and “co-repressor” activity, respectively, which are at least in part recruited by nuclear receptors dependent upon whether they are in the “liganded” or “unliganded” state.

Many of the data concerning co-factors such as co-activators and co-repressor have involved experiments with the retinoid receptors RAR and RXR. Such data suggest that co-repressor molecules generally bind nuclear receptors under physiological conditions in the absence of a nuclear receptor agonist, and dissociate from the nuclear receptor in the presence of such an agonist. Likewise, these data indicate, co-activators do not associate with the nuclear receptor in the absence of an agonist, but are recruited by the liganded nuclear receptor. Nuclear receptor antagonists therefore prevent agonism by indirectly preventing recruitment of co-activator and dissociation of co-repressor, while inverse agonists not only act to prevent recruitment of co-activator, but increase binding of co-repressor to nuclear receptor.

While not wishing to be limited by theory, Applicants believe that nuclear receptors are present as dimers bound to their DNA response elements in the intranuclear environment. These dimers may comprise two nuclear receptor monomers of the same family (e.g., RXR homodimers), or may comprise monomer subunits of different families (e.g., RXR:RAR heterodimers). Additionally, each family may have a number of subtypes, such as RARα, RARβ and RARγ; a homodimer may therefore comprise identical or different subtype monomers. The complex formed upon binding of a nuclear receptor dimer to a nucleic acid response element is called the ternary complex.

Applicants have surprisingly discovered that the rate and extent of binding of nuclear receptor co-factors, particularly co-activators and co-repressors, to nuclear receptors is greatly increased upon the prior formation of a ternary complex containing the receptor dimer and a nucleic acid containing the relevant response element, as compared to the rate and extent of binding of such co-factors in the absence of such a nucleic acid. This is thought to be due to the stabilization of the dimer's conformation by binding between the “P-box” of each subunit (i.e., the amino acid sequence region of the DBD of each nuclear receptor subunit that recognizes and binds to a cognate response element half site) and the response element. Additionally, conformational stabilization provided by binding between the leucine zipper moieties of the nuclear receptor subunits is enhanced when the dimer is bound to a response element.

Thus, according to the presently claimed methods the increase in rate and/or extent of cofactor binding to or dissociation with nuclear receptors is so much enhanced that in many cases the association or dissociation of indigenously present cofactors in (their naturally-occurring amounts) can be detected, obviating the necessity of cloning and overexpressing these cofactors within cells. Accordingly, the sensitivity of the presently claimed assay methods is considerably higher than in previous methods in which nucleic acids containing response elements to which the dimer may bind are not included, and in which the transfection of co-factor genes is required.

Other advantages of detecting the ligand-dependent recruitment or dissociation of nuclear receptor co-factors according to the presently claimed methods include the fact that they provide a means to “dissect” the mechanisms of ligand-mediated transcriptional activation or suppression. Standard co-transfection assays provide an indication of the end result of transcriptional regulation by nuclear receptors, but do not enlighten as to the mechanism of transactivation. By knowing which co-factors may be involved in transcriptional regulation by a particular nuclear receptor, ligands may be found that induce recruitment or dissociation of one type or class of co-factor in preference to another type or class. Thus, drugs may be designed whose activity has a greater specificity for a given transcriptional pathway than another, which may result in their having fewer undesired side-effects than current drugs.

For example, ligands can be found that recruit the co-activator SRC-1 in preference to the co-activator ACTR, or vice versa. While SRC-1 is thought to be a co-activator restricted to nuclear receptors, ACTR is believed to be a co-activator of both nuclear receptors and other transcription factors. Both are normally present in limiting but different concentrations within the cell; thus a ligand that selectively affects the intranuclear equilibrium of SRC-1 would be expected to also affect the transcriptional activities of other nuclear receptors to which SRC-1 binds—however transcriptional pathways other than those affected by nuclear receptors would probably not be affected. This could well result in a nuclear receptor ligand drug having a restricted range of possible side effects than one that is less selective, or which has selectivity for another co-activator.

By the same token, an agent having slective ACTR-recruiting activity may have therapeutic advantages. ACTR has been implicated in AP1 transactivation as well as interferon-γ-mediated STAT transactivation. Thus selective recruitment of ACTR to a given nuclear receptor may provide anti-AP-1 or anti interferon-γ activity by sequestering the available ACTR. There may also be differences in the transcriptional-promoting activity of co-activators, thus using the present methods one could select a drug that would recruit either a more active or less active co-activator, thereby selectively modulating the therapeutic index of the drug.

Thus, in a basic embodiment, the invention comprises a method of determining whether a compound modulates the transcriptional activity of a nuclear receptor dimer comprising the steps:

1) contacting a first nuclear receptor subunit and an optional second nuclear receptor subunit different from said first nuclear receptor subunit, with;

a) a nucleic acid comprising a response element able to bind both subunits of a nuclear receptor homo- or heterodimer comprising said first nuclear receptor subunit and said second nuclear receptor subunit, if present;

b) a compound comprising a prospective ligand of said first nuclear receptor subunit; and

c) a nuclear receptor co-factor capable of directly or indirectly binding either said first nuclear receptor subunit, or said second nuclear receptor subunit, if present, in a ligand dependent manner; and

2) detecting the association or dissociation of said co-factor with said first or second nuclear receptor subunit in the presence of said compound when compared to performing step 1) in the absence of said compound, as an indication that said compound modulates the transcription regulatory activity of said first or second nuclear receptor subunit.

Such an assay may be performed using mammalian cells stably or transiently transfected with one or more expression vector expressing the first and optional second nuclear receptor subunit(s), which cells also express a nuclear receptor co-factor which will directly or indirectly bind to or dissociate from at least one of the nuclear receptors in a ligand dependent manner. According to the invention, the nuclear receptor co-factor may be indigenously expressed or may be expressed as the result of transfection. Methods of transfection, including methods of stably transfecting cells with nucleic acids encoding nuclear receptors and or co-factors are routine and are well known by those of skill in the art. In a preferred embodiment, the nuclear receptor co-factor is indigenously expressed by the cells. Suitable host cells responsive to a given nuclear receptor are well known in the art; for example, for the study of RAR and/or RXR, the human embryonic kidney cell line HEK293, the human cell line HeLa and Green Monkey kidney cell line CV-1 have been commonly used in transactivational assays, and are suitable for the present methods as well.

In a preferred embodiment, the nuclear receptor subunits used in the invention are full length nuclear receptor monomer subunits. However, recombinant subunits comprising truncated or mutated versions of the nuclear receptors and which comprise at least 1) a DNA-binding domain, and 2) a ligand binding domain may be used if desired. It will be appreciated that chimeric receptor subunits having a DNA binding region derived from one nuclear receptor and a ligand-binding domain of another nuclear receptor may be used, particularly to eliminate artifacts caused by the activity of endogenous nuclear receptors.

Likewise, co-factors are preferably endogenous to the cell within which the nuclear receptors are produced. However, such co-factors may be cloned and expressed within the host cell. Additionally, the recombinant co-factor may be present as a mutated or truncated version. In order to function in the present assay the co-factor must 1) have at least one receptor binding region, and 2) associate or dissociate from the receptor in a ligand dependent manner. In one embodiment, the receptor binding region is an LXD found within a receptor interaction domain, as described in further detail below.

The cells are cultured to permit expression of the nuclear receptor(s). Following such expression, the cells may be lysed and a cell-free extract used for the subsequent experiments. Any suitable lysis procedure, including sonication, homogenization and freeze-thawing may be employed; however, the lysis procedure must be sufficient to rupture cell nuclei. The cell-free extract, containing the expressed nuclear receptor(s) and the nuclear receptor co-factor may be used immediately or may be stored at -20° C. or -80° C. until desired.

When performing the assay, the cell-free extract is given an appropriate amount of a nucleic acid response element which will bind a dimer of the nuclear receptor(s) subunits which were expressed within the cells prior to lysis. If only one nuclear receptor was expressed within the cell, the response element will be one able to bind a homodimer of that nuclear receptor. If more than one nuclear receptor was expressed within the cell, the response element will be able to bind at least one homo- or hetero-dimer comprising such nuclear receptor subunits.

The nucleotide sequences of diverse response elements having selectivity to members of the nuclear receptor superfamily have now been determined; additionally, it is to be anticipated that other such response element nucleotide sequences will be determined in the future. The person of skill in the art would easily be able to find published disclosures of these sequences. Additionally, the determination of a specific response element can be determined using routine methods such as nuclease protection method whereby receptor-bound genomic DNA is treated with a deoxyribonuclease, then PCR amplified and the nucleotide sequence of the protected sequence is determined.

In addition to those RE sequences specifically disclosed elsewhere in this application, a RE for FXR is given in Willy, P. W. et al., Gene Dev. 11:289-298 (1997), and a RE for FXR is given in Forman, B. M. et al., Cell 81:687 (1995).

Some response elements may be specific for a given nuclear receptor dimer type, such as RAR/RXR; RXR/RXR; PPAR/RXR; ER/RXR and the like, in which one subunit is invariably from a given member of the superfamily, and the other subunit is also invariably from the same or a different member). However, other response elements may be more promiscuous, having a selectivity that includes more than one dimer type. A nucleic acid comprising at least one such response element, specific or selective for a nuclear receptor expressed within a cell as set forth above, may be used in the assay described and claimed herein.

Similarly, the amino acid sequences of many nucleic receptor monomers (or subunits) are now known; additionally a common nuclear receptor domain which is responsible for the recognition of a receptor's cognate response element half site, termed the “P-box”, has now been characterized, and can be found in the nucleotide sequence of the nuclear receptors.

Specific methods for detecting the association or dissociation of the nuclear receptor co-factor are also diverse. For example, one such format would include methods such as immunoprecipiation of the ternary complex and any associated proteins with an antibody having specificity to a nuclear receptor monomer component thereof from the mixture of prospective ligand, nucleic acid, and cell-free lysate. The immunoprecipitate is then subjected to polyacrylamide electrophoresis and the separated proteins transferred to a suitable membrane for immobilization, such as nitrocellulose, and probed with an antibody having specificity for the nuclear receptor co-factor. When compared to an immunoprecipitate from a lysate similarly treated but not given the prospective ligand, an increase or decrease in the amount of the nuclear receptor co-factor detected by the antibody is an indication that the prospective ligand modulates the transcriptional activity of the nuclear receptor dimer.

In another embodiment, the method of detecting the transcriptional activity of the nuclear receptor may include incubating together the ternary complex and the prospective ligand, then permitting the ternary complex and any associated proteins to specifically bind a solid support. The immobilized complex is then washed, and presented with an second antibody having binding specificity to the nuclear receptor co-factor sought to be identified. This second antibody is then detected; an increase or decrease in the amount of the co-factor sought to be identified (as compared to a control mixture given no prospective ligand) is an indication that the prospective ligand modulates the transcriptional activity of the nuclear receptor dimer.

This latter process is easily reduced to automated methods. For example, the cell-free lysate and nucleic acid response element may be incubated with a panel of test compounds in separate wells of a microtiter dish (such as 96 well plates), then transferred via a robotic pipetting device to a fresh microtiter dish containing wells having an interior surface coated with an antibody specific for one of the two nuclear receptor subunits of the dimer. Washing can be performed by automated pipetting and shaking or mixing of the microtiter dishes. Similar to an ELISA (enzyme-linked immunosorption assay) format, a secondary labeled antibody is then added to each well using the automated pipetting device, the antibody permitted to bind, and then the well washed free of unbound label. In ELISA, the secondary antibody is an enzyme which is then added, permitted to react with a chromogenic substrate, and then detected using a spectrophotometer.

Alternatively, the label may be any moiety capable of detection; these include, without limitation, radioisotopes, luminescent compounds (including chemiluminescent compounds such as acridinium esters and their derivatives), fluorescent compounds, biotin, iminobiotin, avidin, an electron dense component, a magnetic component, an enzyme, a hormone component, or a metal-containing component. Methods of detecting such labels may include, without limitation, spectrophotometry, luminometry, nuclear magnetic resonance, autoradiography, scintillation counting and the like.

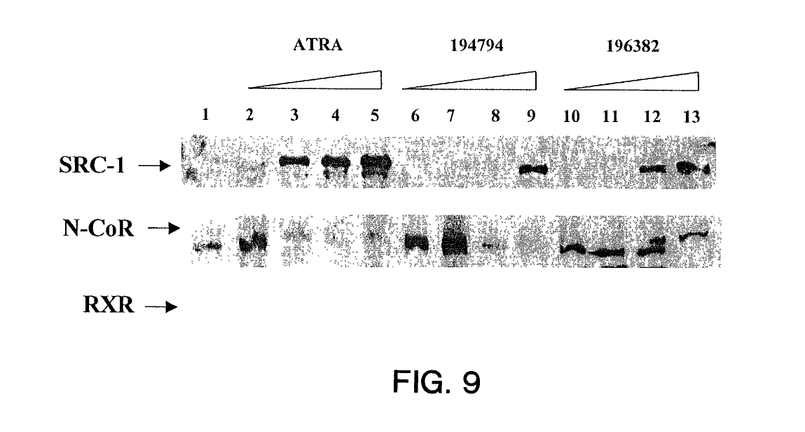

As disclosed herein in Example 9, the DNA dependent coregulator recruitment assay described above was used to measure the ability of RARα selective ligands to recruit the coactivators SRC-1 and ACTR to RARα. As shown in FIG. 9, ACTR recruitment in response to the different ligands was relatively similar, with 194365, 194794 and 196382 resulting in 82-88% recruitment compared to TTNPB, and 196412 resulting in 65% recruitment relative to TTNPB. In contrast, the amount of SRC-1 recruitment was far more divergent in response to the different ligands. In particular, AGN194365 provided a similar degree (65%) of SRC-1 recruitment compared to TTNPB, while SRC-1 recruitment by the remaining compounds was considerably weaker, ranging from 23 to 32% relative to TTNPB. These results indicate that the compounds AGN194794, 196382 and 196412 recruit significantly more ACTR as compared to SRC-1. Thus, these ligands are coactivator selective in that they preferentially recruit ACTR to the RARα receptor as compared to SRC-1. Moreover, the relative ratios of ACTR to SRC-1 recruited in response to a particular ligand are reproducible.

Based on this discovery, the present invention provides a method of identifying a coactivator-selective compound. The method includes the steps of contacting a first nuclear receptor subunit and an optional second nuclear receptor subunit different from the first nuclear receptor subunit, with a nucleic acid containing a nuclear receptor response element able to bind both subunits of a nuclear receptor dimer containing the first nuclear receptor subunit and the second nuclear receptor subunit, if present; a compound containing a prospective ligand of the first or optional second nuclear receptor subunit, if present; and first and second nuclear receptor coactivators which each directly or indirectly bind either the first nuclear receptor subunit, or the second nuclear receptor subunit if present, in a ligand dependent manner; and detecting the association of the first coactivator and the second coactivator with the first or second nuclear receptor subunit in the presence of the compound when compared to performing step 1) in the absence of the compound, where a different extent of association of the first coactivator as compared to the second coactivator indicates that the compound modulates transcription by selectively recruiting one coactivator in preference to another coactivator.

In one embodiment, the contacting step is performed in vitro. In another embodiment, during the contacting step, the first nuclear receptor subunit, optional second nuclear receptor subunit, and nuclear receptor co-factor are contained in a cell lysate. Such a cell lysate can be prepared, for example, from cells transfected with at least one nucleic acid vector expressing the first and the optional second nuclear receptor subunit, if present. In one embodiment, the first or second nuclear receptor coactivator is endogenously expressed by cells from which the cell lysate is made.

A variety of nuclear receptor subunits can be used in a method of the invention for identifying coactivator-selective compounds. A first nuclear receptor subunit can be, for example, RAR, RXR, ER alpha, ER beta, VDR, PPAR, the thyroid receptor, FXR, LXR, the insect ecdysone receptor, the glucocorticoid receptor, the androgen receptor, the progestin receptor, the mineralcorticoid receptor or the CarB receptor. In one embodiment, the first nuclear receptor subunit can be, for example, an RAR subunit, an RXR subunit, an ER alpha subunit, an ER beta subunit, a VDR subunit, a PPAR subunit, a thyroid receptor subunit, an FXR subunit, an LXR subunit, or an insect ecdysone receptor subunit, and the second nuclear receptor subunit can be an RXR subunit. In another embodiment, the first nuclear receptor subunit is an RAR or RXR subunit, and the second nuclear receptor subunit is an RXR subunit. A first nuclear receptor subunit also can be part of a homodimer and can be, for example, a glucocorticoid receptor subunit, an androgen receptor subunit, a progestin receptor subunit, or a mineralcorticoid receptor subunit.

The first and second nuclear receptor coactivators can be, for example, one of the following: SRC-1, N-CoA2, TATA box binding protein (TBP), Creb binding protein (CBP) or ACTR. In one embodiment, the first nuclear receptor coactivator is SRC-1, and, in another embodiment, the first and second nuclear receptor coactivators are SRC-1 and ACTR.

In a method of the invention, the detecting step can include, for example, separating the dimer and any associated first and second nuclear receptor coactivators from other components present during the contacting step and detecting the presence or absence of the first and second nuclear receptor coactivators co-separating with the dimer. The separating step can include, for example, selectively adsorbing the nuclear receptor dimer and any associated first and second nuclear receptor coactivators to an affinity reagent, and determining the presence or absence of first and second nuclear receptor coactivators co-adsorbing with the dimer. An affinity reagent useful in the invention can include, for example, an antibody that selectively binds the dimer.

As used herein, the term “nuclear receptor coactivator” means a protein capable of binding directly or indirectly to a nuclear receptor in a ligand-dependent manner and which exhibits increased binding upon agonist treatment. Where a method of the invention is practiced with first and second coactivators, it is understood that said first and second coactivators are different. Exemplary first and second coactivator molecules useful in the invention are, without limitation, SRC-1, N-COA2, TATA box binding protein (TBP), CREB binding protein (CBP) and ACTR.

A variety of means are described herein above for detecting the association of a co-factor with a first nuclear receptor subunit or second nuclear receptor subunit; such means also are useful for detecting the association of the first and second coactivators in a method of the invention for identifying a coactivator-selective compound. It is understood by those skilled in the art that the association of the first coactivator can be detected before, during, or after detection of association of the second coactivator, and that the association can be detected by the same or different means, as desired.

In a method of the invention, a different extent of association of the first coactivator as compared to the second coactivator indicates that the compound modulates the transcriptional activity of the nuclear hormone receptor by recruiting one coactivator in preference to another coactivator. The extent of association of the first coactivator generally is at least 10% increased or decreased as compared to the second coactivator, and can be at least 20%, 30%, 50%, 100%, 2-fold, 5-fold, 10-fold, 20-fold increased or decreased as compared to the second coactivator.

In one embodiment, the ratio of the association of the first coactivator in the presence of the prospective ligand is determined relative to its association in the presence of a known agonist. Similarly, the association of the second coactivator in the presence of the prospective ligand is determined relative to its association in the presence of the same known agonist. A difference in the relative associations indicates a difference in the extent of association and, therefore, that the prospective ligand is a coactivator-selective compound.

It will be understood that the embodiments of the invention summarized above, and those included in the Examples that follow, are exemplary only and are not intended to limit the scope of the invention, which is defined by the claims that conclude this specification.

### EXAMPLE 1

AGN 192620 and 194204 are RXR specific retinoid agonists of structures:

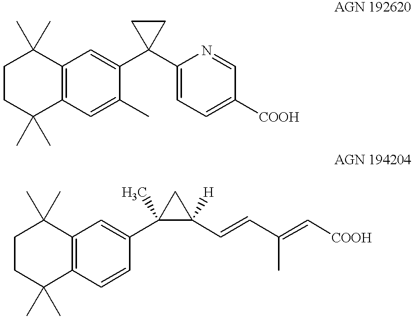

These compounds have binding affinity for the three RXR subtypes but no measurable binding affinity for the three RAR subtypes. Binding is determined as follows. Retinoid receptors (RAR α, β, γ and RXR α, β, γ) are expressed using a Baculovirus expression system, and protein extracts are made. Christensen, K. et al., Molecular Endocrinology 5:1755-1770 (1991), hereby incorporated by reference herein. Stock solutions of the tested compounds are prepared as 10 mM solutions in ethanol. Serial dilutions are made using 1:1 DMSO/ethanol. The ligand binding assays are performed in a solution consisting of 8 mM Tris-HCl (pH 7.4), 120 mM KCl, 4 mM DTT, 8% glycerol, 5 mM CHAPS and 0.24 mM PMSF.

The final test volume of 250 μl contains 10-40 μg of baculovirus extract protein, 5 nM of [³H] all-trans retinoic acid (for the RARs) or 10 nM [³H] 9-cis retinoic acid (for the RXRs), and varying concentrations of competing ligand (0-10⁻⁵M). Incubations are carried out at 4° C. until equilibrium is achieved.

At the end of the incubation period, 50 μl of 6.25% hydroxyapatite is added to wash buffer (100 mM KCl, 10 mM Tris and either 5 mM CHAPS [RXRs] or 0.5% Triton X-100 [RARs]). The mixture is vortexed and incubated for 10 min at 4° C., centrifuged, and the supernatant removed. After rinsing the pellet three times with wash buffer, the amount of receptor-ligand complex is determined by liquid scintillation counting of the hydroxyapitite pellet. After correcting for non-specific binding, the K_{I} value, defined as the concentration of competing ligand required to decrease specific binding by 50%, is determined graphically from a log-logit plot of the data. K_{d} values are determined by application of the Cheng-Prussof equation (Cheng Y-C & Prussof, W. H. Biochemical Pharmacology 22:3099-3108 (1973)).

Binding affinity, as determined by competitive binding experiments, is shown in Table 1; the association constant K_{I} is the compound concentration (in nM) at which half of the receptors are bound to the indicated compound:

TABLE 1

| | RARα | RARβ | RARγ | RXRα | RXRβ | RXRγ |
|---|---|---|---|---|---|---|
| Compound | K_{I} | K_{I} | K_{I} | K_{I} | K_{I} | K_{I} |
| all-trans | 2.4 ± | 2.9 ± | 2.8 ± | ND | ND | ND |
| retinoid | 2.5 | 2.7 | 4.8 | | | |
| acid | | | | | | |
| (ATRA) | | | | | | |
| 9-cis RA | ND | ND | ND | 2.2 ± | 46 ± 19 | 9.3 ± |
| | | | | 2.2 | | 6.5 |
| AGN 192620 | NA | NA | NA | 3 ± 1 | 3 ± 1 | 3 ± 1 |
| AGN 194204 | NA | NA | NA | 0.04 ± | 0.4 ± | 0.4 ± |
| | | | | 0.3 | 0.3 | 0.3 |

As the data indicate, AGN 192620 has high binding affinity for the RXRs (K_{I}=3 nM for the three RARs) which is comparable to that of the natural hormone 9-cis retinoic acid. However, AGN 194204 has even greater affinity for the RXRs, with measured K_{I}s which are approximately 10-fold greater than those of AGN 192620.

In agreement with the measured binding affinities of these ligands for the PARs and RXRs, both AGN 192620 and 194204 activate RXRs (but not RARs) in a cotransfection assay in which cells are transfected with the appropriate expression and reporter plasmids (see Table 2). The assay was conducted as follows.

For analysis of ER-RAR chimeric receptor transactivation, 5×10³ green monkey kidney CV-1 cells (per well of a 96 well plate) were transiently transfected using Lipofectamine (Gibco-BRL) according to the manufacturer's instructions with 100 ng of pERE-tk-Luc [containing the estrogen regulated element of the xenopus vittelogenin A2 gene (Klein-Hitpass, L. et al., Cell 46:1053-1061 (1986)) inserted into the plasmid tk-Luciferase (Glass, C. K. et al., Cell 59:697-708 (1989)] and 4.0 ng of the expression vector pECE (Ellis, L. et al., Cell 45:721-732 (1986)) expressing chimeric ER-RAR receptors consisting of the estrogen receptor A/B and DNA binding domains fused to the DEF domain of RAR α, β or γ (Graupner, G. et al., Biochem. Biophys. Res. Comm. 179:1554-1561 (1991.)) After 6 hours of Lipofectamine/DNA treatment, cells were fed with D-MEM (Gibco-BRL) containing a final concentration of 10% (v/v) activated charcoal extracted fetal bovine serum (Gemini Bio-Products). The following day, cells were treated for 18 hours with the ethanol vehicle alone, or vehicle containing increasing concentrations (10⁻¹⁰ to 10⁻⁶ M final) of ATRA, 9-cis RA, or AGN 192620 or AGN 194204. Cells were lysed and luciferase activity was measured using a luciferase reporter assay system (Promega) per the manufacturer's instructions. All determinations were performed in quadruplicate in at least three independent experiments. Mean values were converted to a percentage relative to the mean value determined for 1 uM all-trans retinoic acid. EC₅₀ values were determined after curve fitting using Graph Pad Prism Software (San Diego).

For analysis of RXR receptor transactivation, 5×10³ green monkey kidney CV-1 cells (per well of a 96 well plate) were transfected as described above using 100 ng of the luciferase reporter plasmid CRBPII-tk-Luc, containing 4 copies of the of the RXR-responsive element from the cytosol retinol binding protein II gene (Mangelsdorf, D. J. et al., Cell 66:555-561 (1991)) and 40 ng of the RXR receptor expression vectors pRS-RXRα, pRS-RXRβ or pRS-RXRγ (Allegreto, E. A., J. Biol. Chem. 268:26625-26633.) These expression plasmids contain RXR cDNAs under the control of the Rous Sarcoma Virus long terminal repeat. Transfected cells were dosed with vehicle alone (ethanol) or vehicle containing increasing concentration (10⁻¹⁰ to 10⁻⁶ M final) of either 9-cis RA or AGN 195183. Analysis of luciferase reporter activity was performed as described above.

Results are as follows:

TABLE 2

| | ER- | ER- | ER- | | | |
|---|---|---|---|---|---|---|
| Com- | RARα | RARβ | RARγ | RXRα | RXRβ | RXRγ |
| pound | EC₅₀ | EC₅₀ | EC₅₀ | EC₅₀ | EC₅₀ | EC₅₀ |
| all-trans | 10.1 ± | 0.4 ± | 0.2 ± | ND | ND | ND |
| retinoid | (3.4) | (0.2) | (0.1) | | | |
| acid | | | | | | |
| (ATRA) | | | | | | |
| 9-cis RA | ND | ND | ND | 35.8 ± | 82.4 ± | 16.9 ± |
| | | | | (44.1) | (28.3) | (3.1) |
| AGN | NA | NA | NA | 0.03 ± | 0.4 ± | 0.04 ± |
| 192620 | | | | (0.18) | (1.8) | (0.26) |
| AGN | NA | NA | NA | 0.002 ± | 0.07 ± | 0.003 ± |
| 194204 | | | | (0.002) | (0.04) | (0.006) |

Values represent mean EC₅₀ values (nM)±standard deviation

These assays again were consistent with the binding experiments; AGN 194204 exhibits an approximately 10-fold greater potency in activation of the RXRs as compared to that of AGN 192620.

However, in both binding and co-transfection assays these data represent ligand activation of homodimers only. Specifically, the RXR transactivation assay represents ligand activation of RXR homodimers and the RAR assay represents ligand activation of chimeric ER-RAR homodimers. Thus, these assays do not measure activity through the naturally occurring RAR/RXR heterodimer.

In order to determine the behavior of the RAR/RXR heterodimer, we measured the transactivation ability of AGN 192620 and AGN 194204 on transfected RXR/RAR-P-GR receptors. The RAR-P-GR receptors are recombinant chimeric receptors in which the P-box for RAR (EGCKG; SEQ ID NO: 2) has been replaced with a nucleotide sequence encoding the glucocorticoid receptor P-box amino acid sequence GSCKV (SEQ ID NO: 3).

Green monkey kidney-derived CV-1 cells were transfected using the lipofectamine reagent as previously described by Klein et al., J. Biol. Chem. 271:22692-22696 (1996). A Beckman Biomek workstation was used for transfection and treatment of CV-1 green monkey kidney cells. 410³ CV-1 cells/well (96-well microtiter plate) were transiently transfected with 0.14 μg of the reporter plasmid MTV-4(R5G)-Luc containing four copies of the DR-5 RARE R5G of the sequence AGGGTTCACCGAAAGAACAGT (SEQ ID NO:4) inserted into the HindIII site of the plasmid ΔMTV-Luc (Hollenberg et al., Cell 55:899 (1988), 0.002 ug of the plasmid pRS-hRXRα (Giguere et al., Nature 330:624 (1987)) and 0.01 ug of pRS-RARα-P-GR (Kurokawa et al., Nature 377:451 (1995)). Additionally, transfections were performed where pRS-hRXRα was substituted with pRS-hRXRβ(Benbrook et al., Nature 333:669 (1988)) or pRS-RXRγ(Ishikawa et al., Mol. Endocrinol. 4:837 (1990)).

Eighteen hours following transfection, cells were rinsed with phosphate buffered saline (PBS) and fed with Dubecco's Modified Eagle's Medium (DMEM; Gibco-BRL) containing 10% activated charcoal extracted fetal bovine serum (Gemini Bio-Products). Cells were treated for 18 hours with either all-trans retinoic acid (ATRA; 10⁻¹⁰ to 10⁻⁶ nM), AGN 192620 (10⁻¹⁰ to 10⁻⁶ nM) or AGN 193109 (10⁻¹⁰ to 10⁻⁶ nM).

AGN 193109 has the structure:

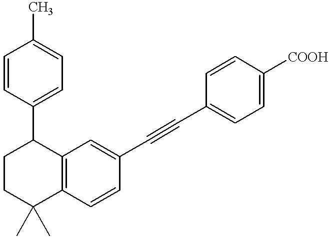

After rinsing the cells with PBS, they were lysed, and luciferase activity was measured in a manner similar to that described by de Wet et al., Mol. Cell. Biol. 7:725 (1987), using firefly luciferin and an 96-well plate luminometer. Luciferase values represent the mean±SEM of triplicate determinations normalized to the maximum ATRA activity.

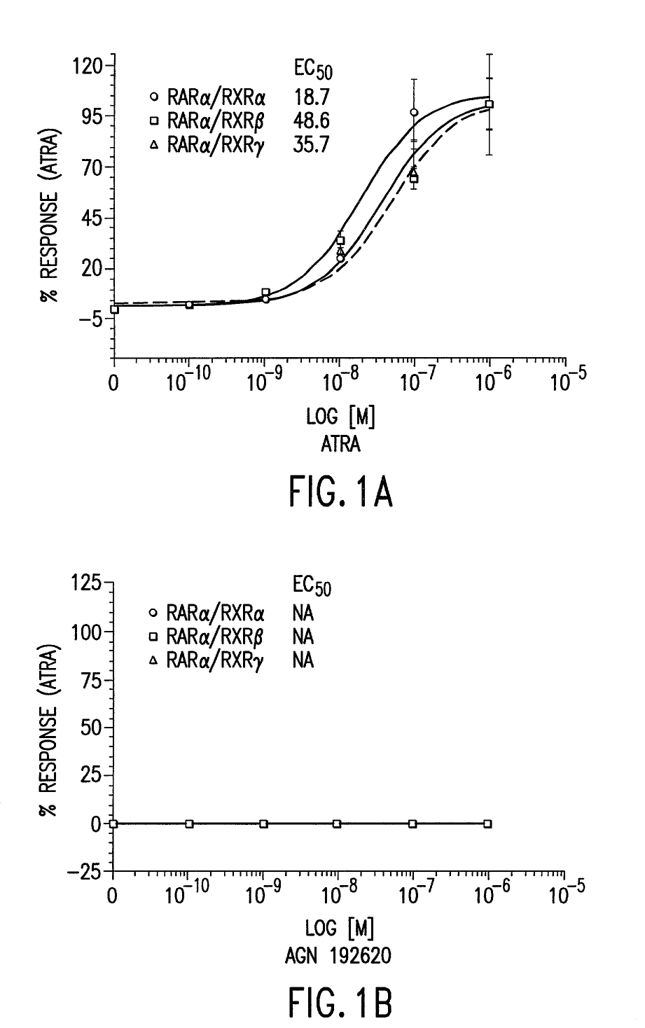

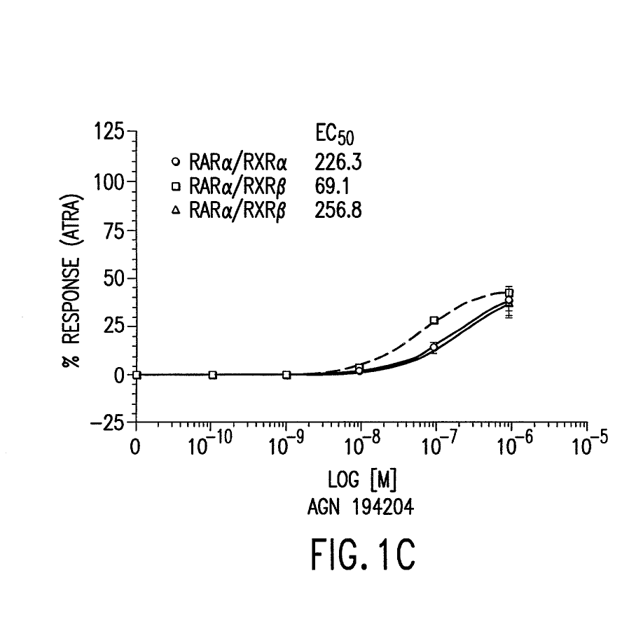

As shown in FIG. 1A, ATRA caused expression of luciferase activity at EC₅₀ values (concentration at which half the maximal transcriptional stimulation occurs) in the 20 to 40 nM range, depending on the identity of the RXR transfected. These EC₅₀ values are in good agreement with the known affinity of this natural hormone for RARα.

As shown in FIG. 1B, AGN 192620, as an RXR-specific agonist, did not stimulate the production of measurable luciferase activity from RXR/RARα-P-GR heterodimers. Surprisingly however, AGN 194204, another RXR specific compound, displayed measurable transactivation ability in this assay, with EC₅₀ values ranging from 69 nM to 257 nM depending on the RXR subtype with which it was co-transfected; see FIG. 1C. These EC₅. values are not in agreement with the measured K, of this compound for the RXRs, or with the measured EC₅₀ values for the activation of RXR homodimers.

Thus, this example shows that AGN 194204 appears to be capable of activating the transcriptional activity of an RAR/RXR heterodimer, albeit at concentrations which are approximately four orders of magnitude greater than those which are sufficient for binding and activation of RXR homodimers.

### EXAMPLE 2

As Example 1 indicated that AGN 194204 treatment resulted in transactivation of RAR/RXR heterodimers, we sought to determine whether treatment of cells with this RXR specific ligand would recruit the nuclear receptor coactivator SRC-1 to the RAR/RXR heterodimer. For this purpose, an RAR expression plasmid was constructed using the epitope tagged expression vector pcDNA3.1-V5/HisA (Invitrogen). This expression plasmid is a bacterial/mammalian shuttle vector that encodes, upon insertion of a heterologous gene and expression of the gene, a fusion protein comprising the heterologous protein fused to the V5 epitope.

Plasmid pcDNA3.1-RARβ-V5 was constructed by replacing the RARβ stop codon with a XbaI site in the plasmid pGEM-hRARβΔ5′ using PCR, followed by insertion, after digestion of the PCR fragment with SacI and XbaI, of the resulting SacI-XbaI fragment into the EcoRV and XbaI sites of the plasmid pcDNA3.1-V5/HisA. pGEM-hRARβΔ5′ is a vector containing human RARβ, but missing the 5′ untranslated nucleotide sequence; the presence or absence of this 5′ untranslated region is not important to practicing this invention. Thus, pcDNA3.1-RARβVS expresses RARE as a fusion protein with the V5 epitope expressed in frame at the C-terminus, affording detection using an anti-V5 monoclonal antibody and normal Western blot analysis. Further, whole cell extracts made from CV-1 cells cotransfected with expression plasmids encoding at least one V5 tagged nuclear receptor (such as pcDNA3.1-RARβ-V5 and pRS-RXRα) can be treated with receptor ligands followed by immunoprecipitation with anti V-5 antibody. The resulting immunoprecipitate can then be probed with a specific antibody in an effort to detect recruitment of the nuclear receptor co-activator SRC-1 in a ligand dependent manner. A consensus DR-5 RARE nucleotide sequence for RAR/RXR heterodimer binding is the double-stranded version of the sequence AGGTCANNNNNAGGTCA (SEQ ID NO: 1; previously disclosed) and its complementary sequence, where the AGGTCA half sites are separated by a spacer of 5 base pairs. Other sequences selective for a given nuclear receptor dimer can suffice and are known to those of skill in the art.

As indicated above, members of the nuclear receptor superfamily have been demonstrated to be modular in nature, as exemplified by the RARs and RXRs, which can be separated into modular domains based upon amino acid similarity and function. For the RARs, these consist of divergent amino-terminal A and B domains followed by a highly conserved C domain. This domain is cysteine rich and encodes so-called zinc-fingers which are responsible for binding to DNA. After the C domain is the D-E-F domain which encodes the carboxyl terminus which provides the following functions: receptor dimerization, ligand binding, transactivation, and interaction with co-regulator molecules, both positive and negative. As such, RAR/RXR heterodimers bind to DNA such that the C-domain of the RAR binds one half-site of the RARE while the C-domain of the RXR binds the other. In certain cases we tested whether addition of a nucleic acid RARE element to the cell lysate containing the human RARβ-V5 and RXRα subunits prior to immunoprecipitation would facilitate the detection of SRC-1 recruitment to RARβ-V5 upon addition of either the RAR agonist TTNPB or the RXR agonist AGN 194204.

Green monkey kidney CV-1 cells were cultured with DMEM containing 10% activated charcoal extracted fetal bovine serum (FBS) before transfection. At a density of 40-60% (15-cm plate, Falcon), cells were transiently transfected with 15 ul FuGene 6 Transfection Reagent (Boehringer Mannheim) with 0.5 ug of pRS-RXRα, and 5 ug of pcDNA3.1-hRARβ-V5 per plate. After two days, cells were rinsed (2×) with PBS and lysed in cold NET buffer (20 mM Tris-Cl [pH8.0], 200 mM NaCl, 1 mM ethylenediamine tetraacetic acid (EDTA), 0.1% NP-40® surfactant, 10% glycerol) containing effective amounts of the protease inhibitors:

4-(2-aminoethyl)benzenesulfonyl fluoride, pepstatin A, trans-epoxysuccinyl-L-leucyl-amido (4-guanido)butane, bestatin, leupeptin and aprotonin, homogenized by QIAshredder (Qiagen), and clarified by centrifugation. Each cell lysate was incubated with 1 uM of the ligand on ice for 1 hour.

Where indicated, an annealed double-strand DR-5 RARE oligonucleotide of the nucleotide sequence

AGCTTTCAGGTCACCAGGAGGTCAGAA (SEQ ID NO: 5)

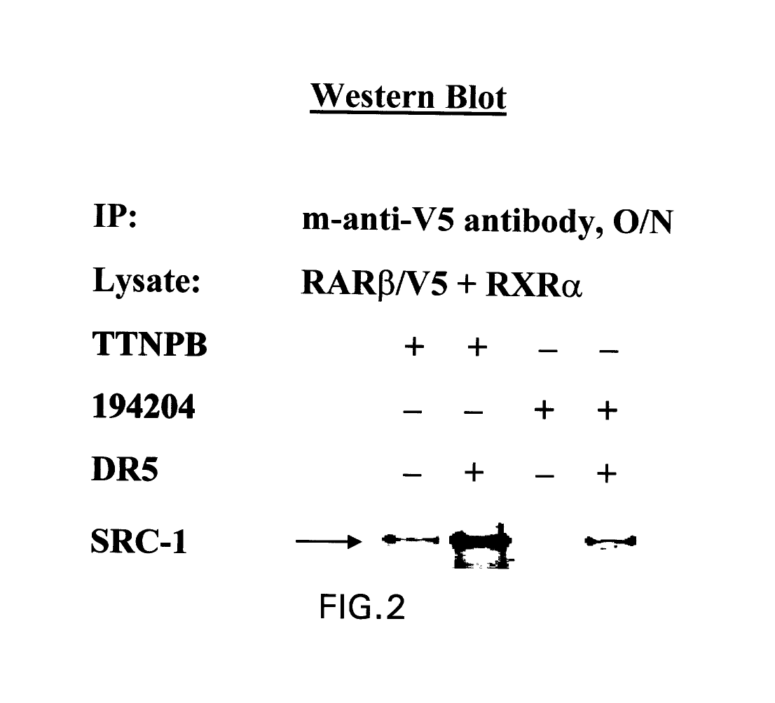

and its annealed complementary strand was added (500 ng/ml final concentration) prior to ligand addition and incubated on ice for 30 minutes. After 1-hour incubation on ice with an anti V5 antibody (mouse anti-VS, Invitrogen), Protein G-agarose (Sigma) was added to the tubes, which were then rocked overnight at 4° C. After washing with ice cold NET buffer, immunoprecipitates were resolved on SDS-polyacrylamide gradient gels (4-12%) followed by Western blotting using standard procedures. The blotted membranes were probed with the indicated antibodies in PBS-T buffer (PBS with 0.1% Tween-200 surfactant) containing 5% nonfat dry milk, and washed in PBS-T buffer. Detection of co-immunoprecipitation of SRC-1 was performed using a mouse anti-SRC-1 monoclonal antibody (Affinity BioReagents #MAl-840) and a horseradish peroxidase(HRP)-linked secondary antibody, followed by exposure to an appropriate HRP substrate. As can be seen in FIG. 2, TTNPB treatment led to coimmunoprecipitation (recruitment) of the co-activator SRC-1 and addition of the DR-5 RARE element further increased the recruitment of this co-activator in TTNB-treated cells.

In contrast, AGN 194204 treatment did not result in detectable SRC-1 recruitment if the DR-5 RARE was omitted from the procedure. However, the addition of the DR-5 RARE to lysates from AGN 194204-treated cells resulted in successful detection of the association of the co-activator SRC-1 with RARb-V5 in the immunoprecipitate. The amount of SRC-1 detected in the AGN 194204 assays containing DR-5 was considerably reduced (approximately 30%) compared to treatment with the RAR agonist TTNPB, consistent with the partial (submaximal) agonism of AGN 194204 seen in Example 1. Thus, this experiment shows that addition of a nucleic acid containing an RE to the immunoprecipitation procedure results in increased sensitivity in co-activator recruitment detection.

### EXAMPLE 3

While not wishing to be limited by theory, a possible explanation of the result of Example 2 is as follows. The ligand-mediated increase in co-activator recruitment to the RAR may be the result of the DNA recognition site having a direct effect upon the conformation of the RAR and/or RXR subunit(s) in the RAR/RXR heterodimer. The conformation of the RAR when in solution may not be optimal for co-activator association as compared to when the RAR is constrained by binding to the RARE.

To test this hypothesis, the “DNA modified” immunoprecipitation assay procedure, as outlined in Example 2, was performed in which the amount of DR-5 RARE added was titrated and the effect on co-activator recruitment was assessed. In addition, the amount of RXR heterodimerized with immunoprecipitated RAR was measured and the effect of titrating the DR-5 RARE was similarly addressed. Specificity of the DNA dependent effects was addressed via the use of a mutated DR-5 element, designated “G-5-G” in which the half sites of the DR-5 are mutated from retinoid receptor sites to those of a glucocorticoid receptor response element.

As before, a conventional immunoprecipiation method was used to determine whether the co-activator SRC-1 is recruited by the RAR pan-agonist TTNPB or the RXR-specific agonist AGN 194204. By “pan-agonist” is meant that the compound stimulates transcriptional activity when liganded to any of RARα, RARβ or RARγsubtypes (in an RAR/RXR heterodimer).

Transfection was performed substantially as indicated above. When cells reached a density of 40% -60%, the cells were transiently transfected with expression plasmid pcDNA3.1hRARγ-V5, containing the nucleotide sequence encoding the human RARγ protein, and pRS-RXRα, encoding the RXRα protein.

The pcDNA3.1hRARγ-V5 vector, is analogous to the pcDNA3.1hRARβ-V5 vector used above and expresses the RARγ subunit as a fusion protein with the V5 epitope:

GKPIPNPLLGLDST (SEQ ID NO: 6)

expressed in frame at the C-terminus. As before, pRS-RXRα expresses the full-length human RXRα within mammalian cells.

The GenBank accession number for human RAR alpha is NM_{—}000964, for human RAR beta is X07282, for human RAR gamma is M57707, for RXR alpha is X66223, for RXR beta is X66224 and for RXR gamma is X66225. These sequences can be obtained over the Internet from the World Wide Web at www.ncbi.nlm.nih.gov/, and are expressly incorporated by reference herein.

The CV-1 cells were transiently transfected and lysates prepared as above. Where indicated, a double-stranded oligonucleotide comprising a DR-S RARE was added at concentrations of 100, 250, 500 or 1000 nanograms/ml for thirty minutes on ice prior to incubation of the lysates with the test compound, the pan-agonist TTNPB. The DR-5 RARE used has the nucleotide sequence (from 5′ to 3′, with half sites underlined):

AGCTTTCAGGTCACCAGGAGGTCAGAA SEQ ID NO: 5

The G-5-G control oligonucleotide was constructed comprising a DR-5 response element in which the half sites were mutated to those recognized by the glucocorticoid receptor (from 5′ to 3′, with half sites underlined):

AGCTTAGAGAACACCGAAAGAACACTA SEQ ID NO: 7

and its complementary strand. The double-stranded G-5-G oligonucleotide was added to the assay mixture at a concentration of 500 ng/ml. Both DR-5 RARE and G-5-G oligonucleotides were annealed with their exactly complementary strands prior to being added to the lysate mixture.

Lysates were then incubated with TTNPB at a concentration of 1 μM for one hour on ice. Tubes not given TTNPB were given an equal volume of the dimethylsulfoxide (DMSO) vehicle alone. The structure of TTNPB is as follows:

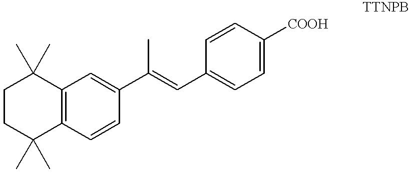

Following incubation with TTNPB, 500 μl the lysates were incubated for a further hour on ice with 1 microliter of a 1 mg/ml stock solution of mouse anti-V5 antibody (purchased from Invitrogen), then each lysate was given 20 μl of a 50:50 (v/v) slurry of Protein G-agarose on NET buffer, and permitted to rock overnight at 4° C.

The agarose beads were washed with ice-cold NET buffer, and the immunoprecipitates were separated by electrophoresis on SDS-polyacrylamide gels (gradient gel; from 4%-12%). Following electrophoresis, the separated proteins were subjected to a Western Blot analysis, conducted by standard methods. Immobilized proteins were probed with an mouse antibody recognizing the SRC-1 co-activator (Affinity Bioreagents, Inc.) As a control, the immunoprecipitated RXRα subunit was detected using a rabbit anti RXRα antibody. The Western blots were developed using an HRP-conjugated secondary anti-mouse IgG antibody, as before.

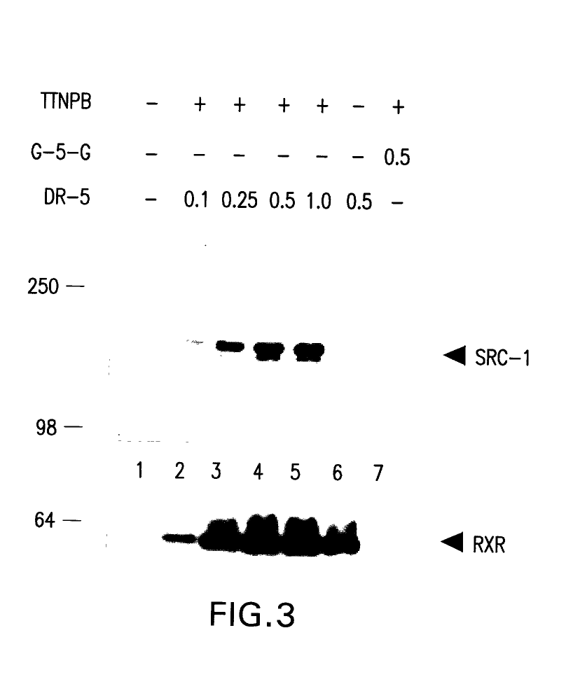

The results are shown in FIG. 3. As can be seen, no SRC-1 recruitment is seen in the absence of added DNA and TTNPB; additionally, only a small amount of RXRU is detected in the immunoprecipitate. Also, no SRC-1 recruitment is seen in the presence of the DR-5 RARE in the absence of TTNPB, as expected, since no transcriptional activation is seen in a co-transfection assay with RAR and RXR in an expression/reporter plasmid system in the absence of an RAR agonist.

The addition of DR-3 RARE in the presence of TTNPB displays a DNA-dependent increase in the amount of SRC-1 recruited. While not wishing to be limited by theory, this appears to suggest that the DR-5 RARE DNA acts to stabilize the conformation of the RARγ/RXRαdimers into one which is necessary for SRC-1 binding. In this assay, at DR-5 concentrations of 250, 500, and 1000 ng/ml, the added DNA aids in the precipitation of RXRU at a constant level. However, there is a clear dose-dependent increase in the amount of SRC-1 detected when the lysates are given 100 through 1000 ng/ml of the DR-5 RARE.

As expected, the addition of 500 ng/ml DR-5 RARE in the absence of TTNPB does not result in the recruitment of the SRC-1 co-activator to the immunoprecipitate. However, when detected with an anti-RXRα antibody, a large increase in the immunoprecipiated RXRα is seen, indicating that addition of DR-5 RARE to the immunoprecipitation reaction mixture aids in the recovery of RAR/RXR heterodimers, probably by increasing the kinetics of dimerization through formation of a ternary complex formation.

Finally, substitution of the G-5-G DNA response element for the DR-5 RARE and addition of this DNA to the lysate tubes in the presence of TTNPB, resulted in a decrease in the amount of both SRC-1 and RXR detected. This finding confirms that the G-5-G response element does not bind or promote formation of the RAR/RXR dimer, and confirms that the increase in assay sensitivity provided by the current invention is nucleotide sequence specific.

It is important to recognize that the SRC-1 detected in this assay was not recombinantly produced, but was endogenously expressed by the CV-1 cells. The present assay may also be used with recombinantly produced co-factors. However, the present invention not only provides for an unexpected increase in the sensitivity of the assays for the detection of recruitment of co-factors to nuclear receptor dimers but, as shown here, it may also be used to permit the detection of naturally produced co-factor without need for overexpression of the protein within the cell by recombinant means. This means that the detected nuclear receptor co-factors are in their native conformation, that they contain any necessary pre- or post-translational modifications, and that they are full-length rather than fusion proteins. Thus, the environment provided by the present assay provides assay conditions that more closely mimic the natural intracellular environment than currently practiced methods.

### EXAMPLE 4

As shown in Example 1, both AGN 192620 and 194204 are RXR-specific ligands as measured both by affinity for the RXRs in vitro as well as in transactivation assays employing transfected retinoid receptors. However, AGN 194204 can be distinguished from AGN 192620 in its ability to activate a RAR/RXR heterodimer bound to a DR-5 RARE element. To determine whether co-activator recruitment is the basis for this apparent difference between these two RXR-specific ligands, we measured the recruitment of the co-activator SRC-1 to the DR-5-bound RARβ/RXRα heterodimer in the presence of AGN 192620 or AGN 194204.

CV-1 cells were transfected with the expression plasmids pcDNA3.1-RARα-V5 and pRS-RXRα substantially as described above. pcDNA3.1-hRARα-V5, was constructed by replacing the human RARα stop codon with a XbaI site in the plasmid pGEM3Z-hRARαΔ5′ using PCR, followed by insertion of the EcoRI-XbaI fragment into the plasmid pcDNA3.1-VS/HisA (Invitrogen). Cells lysates were prepared as described in Example 2. Cell lysates were incubated with vehicle alone or vehicle with the RAR agonist TTNPB (1 μM), the RXR agonist AGN 192620 (1 μM) or the RXR agonist AGN 194204 (1 μM) on ice for 1 hour. A annealed double-strand oligonucleotide consisting of the DR-5 RARE nucleotide sequence SEQ ID NO: 5 and its complementary strand was added to a final concentration of 500 ng/ml prior to ligand addition, and the lysate, oligonucleotide and test compound(s) incubated on ice for 30 minutes. Following this, each sample was incuubated for 1 hour on ice with mouse anti-VS antibody, and immunoprecipiatation with Protein G-agarose conducted as described above. Western analysis of the immunoprecipitates was conducted as described above. Membranes were probed with the indicated antibodies in PBS-T buffer (PBS with 0.1% Tween-20) containing 5% nonfat dry milk, and washed in PBS-T buffer. Detection of co-immunoprecipitated SRC-1 was performed using a mouse anti-SRC-1 monoclonal antibody (Affinity BioReagents #MA1-840) and detection of RXR(was performed using a rabbit anti-RXRα antibody (Santa Cruz Biotech #SC553).

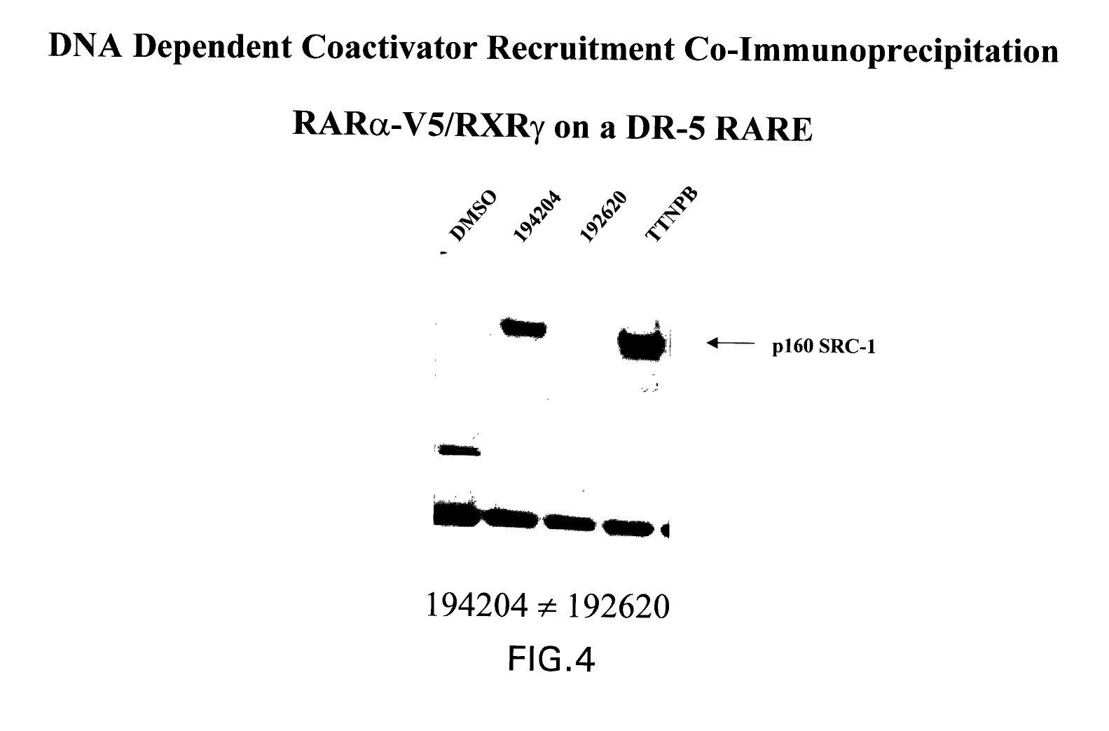

As can be seen in FIG. 4, treatment of the cell lysates with the RAR agonist TTNPB resulted in co-immunoprecipitation of SRC-1, indicating SRC-1 recruitment to the RARα-VS/RXRα/DR-5 ternary complex. Similar to what was found in Example 2, the amount of SRC-1 recruited to the ternary complex upon treatment of the cell lysates with the RXR agonist AGN 194204 was reduced as compared to TTNPB.

In contrast, AGN192620 treatment did not result in detectable SRC-1 recruitment at all.

Thus the differing abilities of AGN 192620 and 194204 to functionally activate RAR/RXR heterodimers in transfected cells shown in Example 1 is here shown to have a corollary: co-activator recruitment is an indication of functional activation of transactivation. The difference in the ability of compounds to activate RAR/RXR heterodimers can be explained by differences in their abilities to recruit co-activators such as SRC-1. Importantly, and as shown in the DR-5 titration experiment described in Example 3, the present method provides heretofor unavailable sensitivity for the detection of co-factor:dimer association. Also, unlike transfection assays in which transfected cells must be incubated with the test compound before the activation is assayed, the present methods have the capability and advantage of being performed in vitro using cell lysates that are previously prepared and stored for later use.

### EXAMPLE 5

Directed mutagenesis within the ligand binding domains of the nuclear receptors has allowed mapping of regions within these domains which are required for transactivation function. With the identification of the various co-activator molecules, it has been demonstrated that receptor regions comprising points of contact between the receptor monomer subunits and the co-activator molecules are required for transactivation. The last of 12 helices within the ligand binding domain of the RXR monomer has been termed the AF2 (“activation function 2”)domain. X-ray crystallographic analysis of the ligand binding domain in RXR homodimers has demonstrated that in the presence of agonist, helix 12 becomes repositioned such that it becomes juxtaposed with helices 4 and 5. This agonist-dependent juxtaposition of helix 12 with helices 4 and 5 results in formation of the co-activator interaction domain. Thus, agonist binding is required for co-activator binding, which in turn is required for functional transactivation. Thus, removal or mutagenesis of helix 12 will result in loss of co-activator interaction.

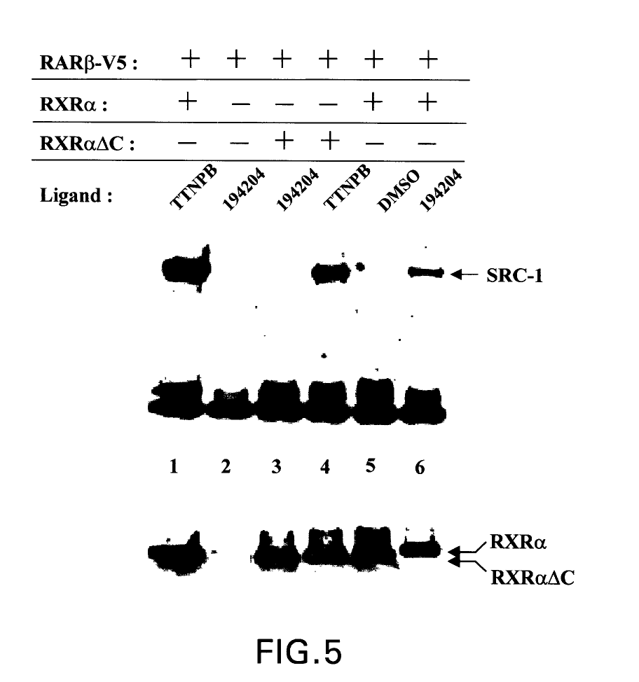

To determine whether AGN 194204 recruitment of SRC-1 to the RAR/RXR/DNA ternary complex reflects recruitment of this coactivator to the RXR component of the complex, we analysed the effect of removal of the RXR AF2 domain by site-directed mutagenesis. CV-1 cells were transfected as previously described with pcDNA3.1RARβ-V5 alone, pcDNA3.1RARβ-VS together with pRS-RXRα, or pcDNA3.1RARβ-V5 together with dnhRXRα (Nagpal S. et al., EMBO J. 12:2349-2360 (1993).) Plasmid dnhRXRα expresses a C-terminal truncated human RXR (called RXRαΔC in FIG. 5) in which the amino acids forming helix 12 are missing. As such, this RXR can no longer interact with co-activator molecules.

Cell lysates were prepared as described above. Cell lysates from pcDNA3.IRARβ-V5 +pRS-RXRα transfected cells were incubated with DMSO vehicle alone or with either the RAR agonist TTNPB (1 μM) or the RXR agonist AGN 194204 (1 μM) on ice for 1 hour. Cell lysates from pcDNA3.1RARβ-VS+dnhRXRα transfected cells were incubated with vehicle, with the RAR agonist TTNPB (1 μM), or the RXR agonist AGN 194204 (1 μM) on ice for 1 hour. Cell lysates from those cells transfected only with pcDNA3.1RARβ-V5 were incubated with DMSO vehicle or with the RXR agonist AGN 194204 (1 μM) on ice for 1 hour. The DR-5 RARE double-strand oligonucleotide used in the previous Examples was added at a final concentration of 500 ng/ml prior to ligand addition and the extracts were incubated on ice for 30 minutes. After 1-hour incubation on ice with mouse anti-V5 antibody, immunoprecipitates were prepared, and subjected to Western analysis as previously described. Membranes were probed with mouse anti-SRC-1 monoclonal antibody and a rabbit anti-human RXRα antibody.

As can be seen in FIG. 5A, treatment with TTNPB (lane 1) leads to recruitment of SRC-1 to the RARβ/RXRα/DR5 ternary complex. In comparison to lane 1, treatment with the RXR agonist AGN 194204 (lane 2) results in a quantitatively reduced SRC1 recruitment. Removal of the C-terminal helix 12 of the RXR subunit of the RAR/RXR heterodimer, however, still results in SRC-1 recruitment to the heterodimer by the RAR agonist TTNPB (lane 4) but the amount of SRC-1 associated with the RAR/RXRαΔC heterodimer upon treatment with AGN 194204 (lane 3) is drastically reduced by comparison. The amount of SRC-1 recruited in the presence of AGN 194204 (lane 3) to the RARβ/RXRαΔC/DR5 ternary complex is comparable to that which is recruited by this ligand from lysates prepared from cells transfected with pcDNA3.1RARβ-V5 only (lane 2). Although not wishing to be limited by theory, this weak signal may reflect the presence of full-length RXRα in heterodimeric association with RARα which is endogenously produced in the CV-1 cells used in these experiments.

Measurement of RXRα associated in the immunoprecipitated complexes (FIG. 5B) indicates very similar amounts of RXRα present in all lanes, with the exception of lane 2, as expected. The faint RXRα signal in lane 2 represents the endogenous RXRα from CV-1 cells. The slightly faster mobility of RXRαΔC in the dnRXRα-transfected lysates reflects the smaller size of this truncated RXR relative to full length RXRα.

These results indicate that the AF2 domain of RXR is required for AGN 194204 recruitment of SRC-1 to the RAR/RXR/DR5 ternary complex. In contrast, RAR agonist-mediated SRC-1 recruitment to this complex does not require the AF2 domain of RXR.

### EXAMPLE 6

We were interested to see if the increased sensitivity provided by the present invention for nuclear receptor agonist-mediated recruitment of co-activator could provide a similar increase in sensitivity for the measurement of co-repressor interaction.

For this purpose, CV-1 cells were transfected with the expression plasmids RS-RXα and pCMV3.1-RARγ-V5, substantially as outlined in Example 2. Whole cell lysates were prepared and incubated with DMSO vehicle alone or vehicle with the RAR inverse agonist AGN 193109 (1 μM), the RAR neutral antagonist AGN 193840 (1 μM) and the RAR agonist TTNPB (1 μM) on ice for 1 hour. The structure of AGN 193840 is as follows:

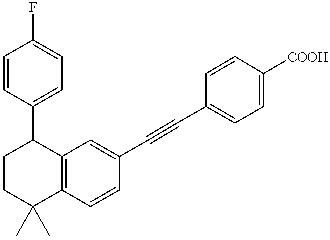

Where indicated, the same double-stranded DR-5 RARE or G-5-G double-stranded oligonucleotides used above were added to a 0.5 μg/ml final concentration prior to ligand addition. Immunoprecipitation with mouse anti-V5 antibody and Western analysis were conducted as set forth above. Detection of the nuclear receptor co-repressor N-CoR which co-immunoprecipitated with the heterodimer upon treatment with RARγ-V5 was performed using a goat anti-N-CoR antibody; detection of RXRα was performed using a rabbit anti-RXRα antibody (Santa Cruz Biotech #SC553).

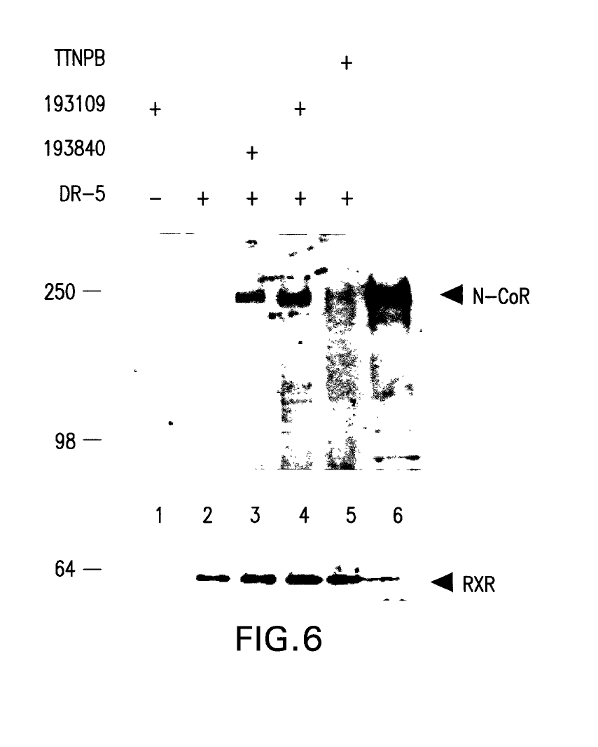

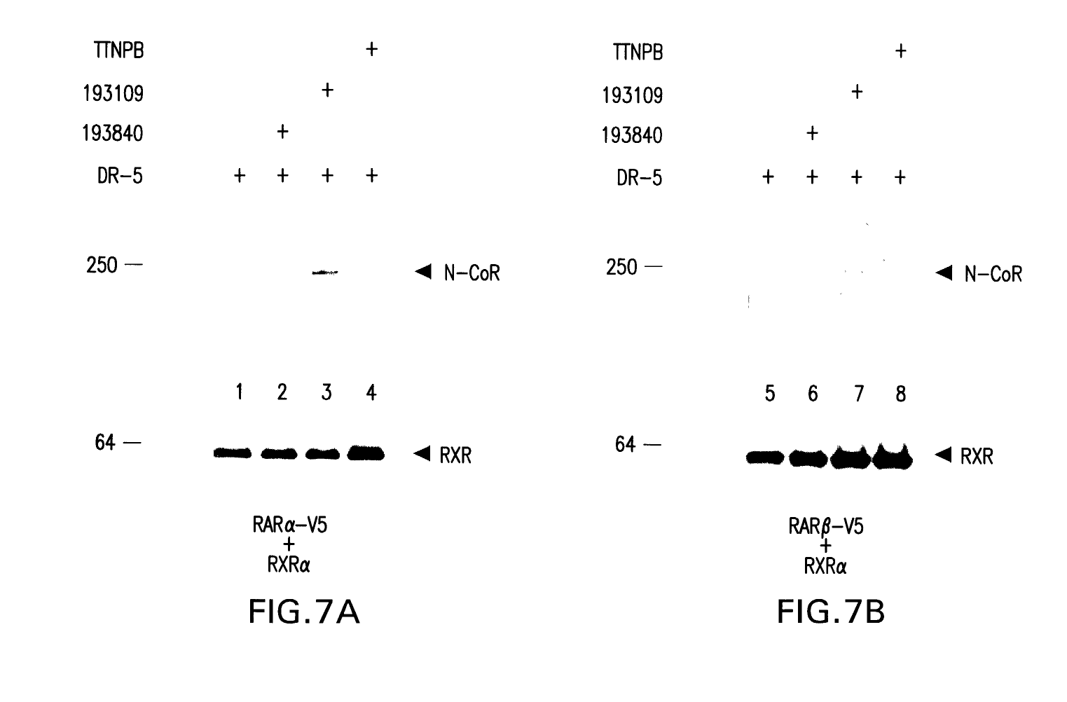

As shown in FIG. 6, in the presence of the DR-5 RARE (lanes 2 to 5) N-COR association with RARα was very weak (lane 2) in the absence of ligand, and not detectable at all after treatment with the RAR agonist TTNPB (lane 5). Both AGN 193840 (lane 3) and AGN 193109 (lane 4) treatment of the cell lysates led to N-COR recruitment, however, these analyses consistently demonstrated a difference in the co-repressor recruitment capability of these two antagonists. AGN 193109 treatment resulted in an approximately two-fold increase in N-CoR recruitment as compared to the amount of N-COR recruited upon treatment with AGN 193840.

Our ability to detect the association of N-COR with the RAR/RXR heterodimer upon lysate treatment with AGN 193109 required the presence of a DR-5 RARE nucleotide sequence, since use of a related G-5-G DNA element (lane 1) with AGN 193109-treated cell lysates failed to result in the detection of co-repressor. Therefore, in a manner similar to the use of the invention in RAR-co-activator analyses, this method also allows for increased sensitivity in the analysis of RAR-co-repressor interactions.

This fact is consistent with a model in which the DR-5 nucleic acid provides a “nucleation” zone for formation of nuclear receptor dimerization, resulting in an acceleration of dimer formation and reconstitution of a more natural nuclear receptor conformation in vitro during the immunoprecipitation process. As shown using RAR/RXR heterodimers as a model system, this more natural conformation has a more avid affinity for nuclear receptor co-factors, such as the co-activator SRC-1 and (in the presence of an inverse agonist) the nuclear receptor co-repressor N-CoR than is the case when no nucleic acid response element is added.

This experiment therefore indicates that this “more natural” dimer conformation as a ternary complex has a more avid affinity for the nuclear receptor co-repressor N-CoR in the presence of the RAR inverse agonist AGN 193109 than compared to RARγ-V5 in the absence of added DR-5 RARE. The previous experiments show that the same increase in affinity is seen for co-activators using the presently claimed methods. Because co-factors modulating nuclear receptor transcriptional activity are known or believed to exist for many of the nuclear receptors in addition to RAR and RXR heterodimers, and because of the common modular structure and mechanisms of transcriptional action shared by the nuclear receptors, this method has predictable applicability to screening ligands of nuclear receptors for transcriptional activation generally.

### EXAMPLE 7

The domains within the co-activator SRC-1 which are required for interaction with retinoid receptors have been mapped to a central receptor interacting region containing three “LXXLL” domains, wherein L is leucine and X is any amino acid; these domains are termed helical domains or LXDs (see e.g., Torchia, J. et al., Nature 387:677-684 (1997); Heery, D. M. et al., Nature 387:733-736 (1997).) To date, LXDs have been characterized in CBP, p300 (2 LXDs), RIP140 (9 LXDs), SRC-1, ACTR, TIF2 (also called GRIP 1) (3 LXDs in the receptor interaction domain) and TIF1.

The LXDs of SRC-01 and ACTR, termed LXD 1, LXD 2, and LXD 3, are located in a highly conserved Receptor Interaction Domain)(RID), and exhibit an amphipathic helical structure and make direct contact with the co-activator interaction domain of the nuclear receptor formed upon binding of a receptor agonist. Nolte, R. T. et al., Nature 395:137-143 (1998). We addressed the importance of each of the three LXDs in mediating co-activator-receptor interaction using synthetic peptides comprising the LXD1, LXD2 or LXD3 amino acid sequences as competitors with SRC-1 in the DNA modified immunoprecipitation methods of the present invention. These peptides have the following amino acid sequences (with the characteristic LXXLL motif shown in bold):

| | |
|---|---|
| ACTR peptides: | |
| LXD1 (SEQ ID NO: 8): | LESKGHKKLLQLLTCSSDDRGH; |
| LXD2 (SEQ ID NO: 9): | LLQEKHRTLHKLLQNGNSPAEV; |
| LXD3 (SEQ ID NO: 10): | KKKENNALLRYLLDRDDPSDAL |
| SRC-1 peptides: | |
| LXD1 (SEQ ID NO: 11): | KYSQTSHKLVQLLTTTAEQQLR; |
| LXD2 (SEQ ID NO: 12): | SLTERHKILHRLLQEGSPSDIT, |
| LXD3 (SEQ ID NO: 13): | KESKDHQLLRYLLDKDEKDLRS |

Transfection, DR-5 oligonucleotide treatment, ligand treatment, immunoprecipitation and detection of associated co-factors were performed essentially as shown in the previous Examples. CV-1 cells were transfected with plasmids expressing either RARα/RXRγ-V5 or with RARγ/RXRα-V5. Synthetic LXD peptides were added to the cell lysate before the addition of retinoids at a concentration of 20 (ACTR) and 40 (SRC-1) μl.

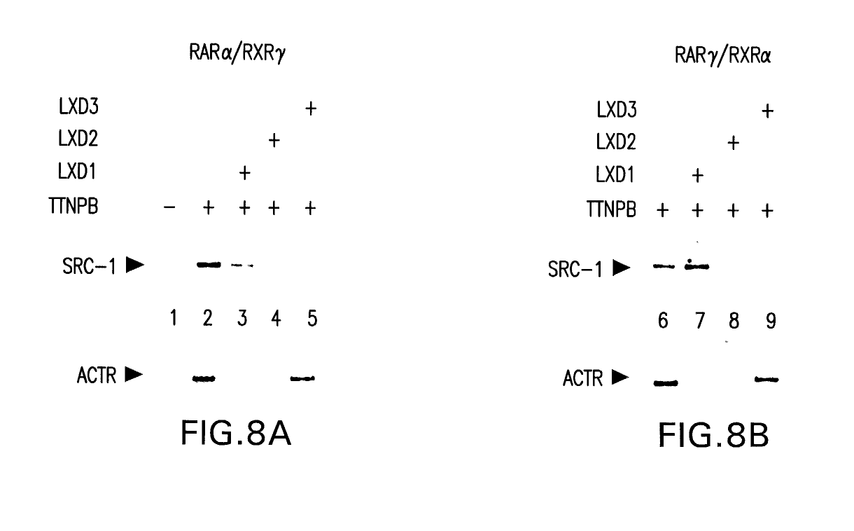

As shown in FIG. 8, ATRA-induced recruitment of SRC-1 to the RARα-V5/RXRγ/DR-5 ternary complex required both the LXD2 and LXD3 peptides, as it was completely abolished by addition of SRC-1 peptides specific for LXD2 and LXD3, whereas addition of the LXD1 peptide had no effect. In contrast, ATRA-induced recruitment of ACTR, another member of the p160 family of nuclear receptor co-activators, to RARα/RXRγ/DR-5 was abolished by addition of competitive amounts of either the LXD1 or LXD2 domains.

Similar results were seen when the synthetic LXDs were added to cell lysates from cells overexpressing RXRα and RARγ (FIG. 8B). However, in this case the amount of SRC-1 shown to associate with the RAR/RXR heterodimer in the presence of LXD1 was somewhat greater than in the previously described experiment.

Thus, while SRC-1 and ACTR exhibit a high degree of amino acid identity within the LXD-containing RID, these related co-activator molecules appear to utilize distinct LXD interfaces for receptor interaction.

### EXAMPLE 8

Various co-activator and co-repressor molecules which interact with members of the nuclear receptor superfamily have now been identified. For a given heterodimer combination, the ability of a nuclear receptor to associate with more than one co-activator or co-repressor molecule in a ligand dependent manner would potentially result in more than one regulatory pathway being activated. It would be preferable to find ligands that are able to activate a given pathway selectively or exclusively to prevent unintended side effects.

For example, the anti AP1 and the transactivation activities of the RAR has been demonstrated to be separable (Fanjul et al. Nature 372: 107-111 (1994) and Nagpal et al., J. Biol. Chem. 270: 923-927 (1995)). Thus, use of the invention to detect ligands which bind to the ternary complex and result in selective co-activator recruitment has the potential to identify ligands with a narrower spectrum of action and hence, increased therapeutic index.

Thus, compound AGN-X is analyzed using the methods of the present invention for its ability to recruit the coactivators SRC-1 and ACTR to the RARβ/RXRα/DR5 ternary complex.

Green monkey kidney CV-1 cells are cultured with D-MEM (Gibco-BRL) containing 10% activated charcoal extracted fetal bovine serum (Gemini Bio-Products) before transfection. At a density of 40-60% (15-cm plate, Falcon), cells are transiently transfected with 15μl FuGene 6 Transfection Reagent (Boehringer Mannheim) with 0.5 μg of pRS-RXRα, and 5 μg of pcDNA3.1-hRARβ-VS per plate. After two days, cells are rinsed (2X) with PBS and lysed in cold NET buffer (20 mM Tris-Cl [pH8.0], 200 mM NaCl, 1 mM EDTA, 0.1% NP-40, 10% glycerol) containing protease inhibitors, homogenized by QIAshredder (Qiagen), and clarified by centrifugation. Cell lysates are incubated with either ATRA, TTNPB or AGN-X (1 μM) on ice for 1 hour. An annealed double-strand oligonucleotide (DR-5 RARE, as used in the previous examples) is added to a final concentration of 500 ng/ml and mixed prior to ligand addition. Mixtures are incubated on ice for 30 minutes, then incubated for 1 hour on ice with primary antibody (mouse anti-V5, Invitrogen). Protein G-agarose (Sigma) is added and samples are rocked overnight at 4° C. After washing with ice cold NET buffer, immunoprecipitants are resolved on SDS-polyacrylamide gels (4-12%) followed by Western blotting as above. Membranes are probed with the indicated antibodies in PBS-T buffer (PBS with 0.1% Tween-20) containing 5% nonfat dry milk, and washed in PBS-T buffer. Detection of co-immunoprecipitation of SRC-1 is performed using a mouse anti-SRC-1 monoclonal antibody (Affinity BioReagents #MA1-840).

Both ATRA and TTNPB treatment lead to coimmunoprecipitation (recruitment) of the co-activators SRC-1 and ACTR to the ternary complex. The amount of SRC-1 detected with AGN-X is equivalent to the amount recruited by ATRA and TTNPB. However, the amount of ACTR recruited by AGN-X is less than 5% than the amount recruited by ATRA and TTNPB. Thus, AGN-X is a coactivator-selective retinoid ligand.

This examples therefore demonstrates the use of the invention to screen for retinoid ligands with co-factor-selective characteristics. By analogy to RAR subtype selective ligands which have a reduced spectrum of action compared to RAR agonists which activate all three of the RAR isoforms, AGN-X has an even narrower spectrum of retinoid action. As such, AGN-X has an enhanced therapeutic index relative to RAR-subtype selective ligands.

### EXAMPLE 9

### Differential Coactivator Recruitment to a Nuclear Hormone Receptor

This example demonstrates differential coactivator recruitment to a nuclear hormone receptor.

Divergent amino acid residues within the ligand binding domains of different RAR isotypes provide a structural basis for the RARα subtype selectivity observed for synthetic retinoid ligands containing an internal amide linkage. RARαπselective ligands shown in Table 3 share this amide linkage structure as well as similar affinities for RARE. However, in contrast to their similar binding affinities, these ligands exhibited disparate transactivation properties at RARα. Specifically, AGN194365 exhibited potent and effective transactivation properties at RARE which were comparable to TTNPB. AGN194794 also activated RARα, albeit with slightly less efficacy and potency. In contrast, AGN196382 and 196412 had no activity at RARα except at the highest dose (1 μM) tested.

TABLE 3
Relative Kds for RARα

| TTNPB | RARα |
|---|---|
| 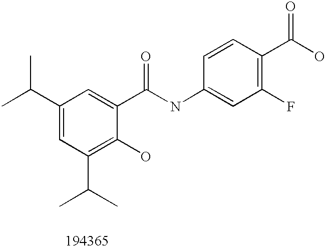 | 3 (37 LG) |
| 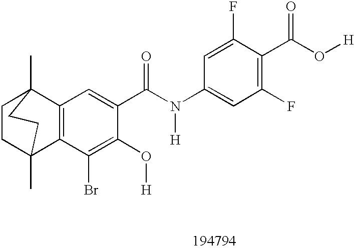 | 69, 99 |
| 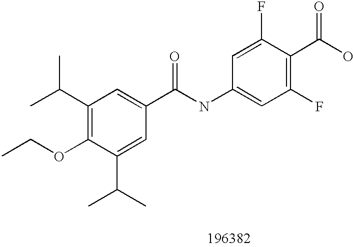 | 27 |
| 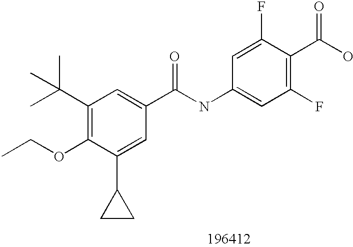 | 6 |

The DNA dependent coregulator recruitment assay described above was used to measure the ability of RARαselective ligands shown in Table 3 to recruit the coactivators SRC-1 and ACTR to the RARα/RXR heterodimer bound to a DR-5 retinoic acid receptor response element (RARE). As expected, association of both coactivator proteins with the ternary complex required ligand (FIG. 3). ACTR recruitment in response to the different ligands was relatively similar, with 194365, 194794 and 196382 resulting in 82-88% recruitment compared to TTNPB, and 196412 resulting in 65% recruitment relative to TTNPB. In contrast, the amount of SRC-1 recruitment was far more divergent in response to the different ligands. AGN194365 provided similar degree (65%) of SRC-1 recruitment compared to TTNPB, while SRC-1 recruitment by the remaining compounds was considerably weaker, ranging from 23 to 32% relative to TTNPB.

The amount of SRC-1 recruited to the ternary complex did not correlate with the transactivation profiles of the compounds. Specifically, the efficiency of SRC-1 recruitment by 194794, 196382 and 196412 was very similar yet AGN194794 was a significantly more efficient transactivator of RARα.

These results indicate that a given ligand can selectively recruit a first coactivator in preference to a second coactivator.

The preceding examples are intended to illustrate certain embodiments of the present invention, and not to limit the scope of the invention in any way. The invention is defined solely by the claims which conclude this specification.

What is claimed is:

1. A method of identifying a compound that modulates the transcriptional activity of a nuclear receptor dimer comprising the steps:
a) contacting a first nuclear receptor subunit and an optional second nuclear receptor subunit different from said first nuclear receptor subunit, with;
i) a nucleic acid comprising a nuclear receptor response element able to bind both subunits of a nuclear receptor dimer comprising said first nuclear receptor subunit and said second nuclear receptor subunit, if present;
ii) a compound comprising a prospective ligand of said first or optional second nuclear receptor subunit, if present; and
iii) a nuclear receptor co-factor which will directly or indirectly bind either said first nuclear receptor subunit, or said second nuclear receptor subunit if present, in a ligand dependent manner; and
b) detecting the association or dissociation of said co-factor with said first or second nuclear receptor subunit in the presence of said compound when compared to performing step 1) in the absence of said compound, as an indication that said compound modulates the transcription regulatory activity of said nuclear receptor dimer.

2. The method of claim 1 wherein said contacting step is performed in vitro.

3. The method of claim 2 wherein during said contacting step said first nuclear receptor subunit, optional second nuclear receptor subunit, and nuclear receptor co-factor are contained in a cell lysate.

4. The method of claim 3 wherein said cell lysate is prepared from cells transfected with at least one nucleic acid vector expressing said first and said optional second nuclear receptor subunit, if present.

5. The method of claim 2 wherein said nuclear receptor co-factor is endogenously expressed by cells from which said cell lysate is made.

6. A method of identifying a compound that modulates the transcriptional activity of a nuclear receptor dimer comprising the steps:
a) contacting a first nuclear receptor subunit and an optional second nuclear receptor subunit different from said first nuclear receptor subunit, with;
i) a nucleic acid comprising a nuclear receptor response element able to bind both subunits of a nuclear receptor dimer comprising said first nuclear receptor subunit and said second nuclear receptor subunit, if present,
wherein said first nuclear receptor subunit is selected from the group consisting of RAR, RXR, ER alpha, ER beta, VDR, PPAR, the thyroid receptor, FXR, LXR, the insect ecdysone receptor, the glucocorticoid receptor, the androgen receptor, the progestin receptor, the mineralcorticoid receptor and the CarB receptor;
ii) a compound comprising a prospective ligand of said first or optional second nuclear receptor subunit, if present; and
iii) a nuclear receptor co-factor which will directly or indirectly bind either said first nuclear receptor subunit, or said second nuclear receptor subunit if present, in a ligand dependent manner; and
b) detecting the association or dissociation of said co-factor with said first or second nuclear receptor subunit in the presence of said compound when compared to performing step 1) in the absence of said compound, as an indication that said compound modulates the transcription regulatory activity of said nuclear receptor dimer.

7. The method of claim 6 wherein said first nuclear receptor subunit is selected from the group consisting of a glucocorticoid receptor subunit, an androgen receptor subunit, a progestin receptor subunit, and a mineralcorticoid receptor subunit, and said nuclear receptor dimer is a homodimer.

8. The method of claim 6 wherein said first nuclear receptor subunit is selected from the group consisting of an RAR subunit, an RXR subunit, an ER alpha subunit, an ER beta subunit, a VDR subunit, a PPAR subunit, a thyroid receptor subunit, an FXR subunit, an LXR subunit, and an insect ecdysone receptor subunit, and wherein said second nuclear receptor subunit comprises an RXR subunit.

9. The method of claim 8 wherein said first nuclear receptor subunit is selected from the group consisting of an RAR subunit and an RXR subunit, and wherein said second nuclear receptor subunit comprises an RXR subunit.

10. A method of identifying a compound that modulates the transcriptional activity of a nuclear receptor dimer comprising the steps:
a) contacting a first nuclear receptor subunit and an optional second nuclear receptor subunit different from said first nuclear receptor subunit, with;
i) a nucleic acid comprising a nuclear receptor response element able to bind both subunits of a nuclear receptor dimer comprising said first nuclear receptor subunit and said second nuclear receptor subunit, if present;
ii) a compound comprising a prospective ligand of said first or optional second nuclear receptor subunit, if present; and
iii) a nuclear receptor co-factor which will directly or indirectly bind either said first nuclear receptor subunit, or said second nuclear receptor subunit if present, in a ligand dependent manner,
wherein said nuclear receptor co-factor is selected from the group consisting of SRC-1, N-CoA1, N-CoA2, TATA box binding protein (TBP), Creb binding protein (CBP) and ACTR; and
b) detecting the association or dissociation of said co-factor with said first or second nuclear receptor subunit in the presence of said compound when compared to performing step 1) in the absence of said compound, as an indication that said compound modulates the transcription regulatory activity of said nuclear receptor dimer.

11. The method of claim 10 wherein one or more of said nuclear receptor cofactors associates to a greater degree with said first or said optional second nuclear receptor subunit in the presence of said compound when compared to performing step 1) in the absence of said compound, as an indication that said compound is an agonist of said nuclear receptor dimer.

12. The method of claim 2 in which said nuclear receptor cofactor is selected from the group consisting of N-CoR and SMRT.

13. The method of claim 12 wherein one or more of said nuclear receptor cofactors associates to a lesser degree with said first or said optional second nuclear receptor subunit in the presence of said compound when compared to performing step 1) in the absence of said compound, as an indication that said compound is an antagonist or reverse agonist of said nuclear receptor dimer.

14. The method of claim 2 wherein said detecting step comprises separating the dimer and any associated nuclear receptor co-factor from other components present during the contacting step and detecting the presence or absence of said nuclear receptor co-factor co-separating with said dimer.

15. A method of identifying a compound that modulates the transcriptional activity of a nuclear receptor dimer comprising the steps:
a) contacting a first nuclear receptor subunit and an optional second nuclear receptor subunit different from said first nuclear receptor subunit, with;
i) a nucleic acid comprising a nuclear receptor response element able to bind both subunits of a nuclear receptor dimer comprising said first nuclear receptor subunit and said second nuclear receptor subunit, if present;
ii) a compound comprising a prospective ligand of said first or optional second nuclear receptor subunit, if present; and
iii) a nuclear receptor co-factor which will directly or indirectly bind either said first nuclear receptor subunit, or said second nuclear receptor subunit if present, in a ligand dependent manner;
b) selectively adsorbing the nuclear receptor dimer and any associated nuclear receptor co-factor to an affinity reagent and
c) determining the presence or absence of co-factor co-adsorbing with said dimer in the presence of said compound when compared to performing step 1) in the absence of said compound, as an indication that said compound modulates the transcription regulatory activity of said nuclear receptor dimer.

16. The method of claim 15 wherein said affinity reagent comprises a first antibody selectively binding said dimer, and said co-adsorbing co-factor is detected using a second antibody.

17. The method of claim 2 wherein said contacting step is performed by automated means.

18. The method of claim 17 wherein said detecting step is performed by automated means.

19. The method of claim 10 wherein said contacting step is performed by automated means.

20. The method of claim 19 wherein said detecting step is performed by automated means.

21. The method of claim 12 wherein said contacting step is performed by automated means.

22. The method of claim 21 wherein said detecting step is performed by automated means.

23. A method for enhancing the detection of a nuclear receptor co-factor associated with a nuclear receptor dimer comprising the steps:
a) contacting said at least one nuclear receptor subunit and said nuclear receptor cofactor with a nucleic acid comprising a nuclear receptor response element which will bind a nuclear receptor dimer comprising said subunit, and
b) detecting the increased association of said nuclear receptor co-factor with said dimer, as compared to conducting step a) in the absence of said nucleic acid.

24. The method of claim 23 wherein said nuclear receptor co-factor detectably associates with said nuclear receptor dimer upon addition of at least one nuclear receptor agonist in said contacting step as compared to an identical method in which a nuclear receptor agonist is not added in said contacting step.

25. The method of claim 24 wherein said nuclear receptor co-factor is selected from the group consisting of SRC-1, N-CoA1, N-CoA2, TATA box binding protein (TBP), Creb binding protein (CBP), and ACTR.

26. A method of identifying a compound that modulates the transcriptional activity of a nuclear receptor dimer comprising the steps:
a) contacting a first nuclear receptor subunit and an optional second nuclear receptor subunit different from said first nuclear receptor subunit, with;
i) a nucleic acid comprising a nuclear receptor response element able to bind both subunits of a nuclear receptor dimer comprising said first nuclear receptor subunit and said second nuclear receptor subunit, if present,
wherein at least one nuclear receptor subunit is selected from the group consisting of RAR, RXR, ER alpha, ER beta, VDR, PPAR, the thyroid receptor, FXR, LXR, the insect ecdysone receptor, the glucocorticoid receptor, the androgen receptor, the progestin receptor, the mineralcorticoid receptor, and the CarB receptor;
ii) a compound comprising a prospective ligand of said first or optional second nuclear receptor subunit, if present; and
iii) a nuclear receptor co-factor which will directly or indirectly bind either said first nuclear receptor subunit, or said second nuclear receptor subunit if present, in a ligand dependent manner; and
b) detecting the association or dissociation of said co-factor with said first or second nuclear receptor subunit in the presence of said compound when compared to performing step 1) in the absence of said compound, as an indication that said compound modulates the transcription regulatory activity of said nuclear receptor dimer.

27. The method of claim 26 wherein said at least one nuclear receptor subunit is selected from the group consisting of the glucocorticoid receptor, the androgen receptor, the progestin receptor, and the mineralcorticoid receptor.

28. The method of claim 26 wherein said at least one nuclear receptor subunit is selected from the group consisting of RAR, RXR, ER alpha, ER beta, VDR, PPAR, the thyroid receptor, FXR, LXR, and the insect ecdysone receptor.

29. The method of claim 26 wherein said detecting step comprises: immobilizing a complex comprising said nuclear receptor dimer associated with said nuclear receptor co-factor, and identifying the presence of said co-factor which co-immobilizes in said complex.

30. The method of claim 29 wherein said identifying step comprises permitting an antibody selective therefor to bind said co-factor, and directly or indirectly detecting the presence of said bound antibody.

31. The method of claim 29 wherein said contacting and immobilization steps take place in a microtiter-type vessel.

32. The method of claim 30 wherein said contacting and immobilization steps take place in a microtiter-type vessel.

33. The method of claim 23 wherein said nuclear receptor co-factor detectably dissociates from said nuclear receptor dimer upon addition of at least one nuclear receptor antagonist or inverse agonist in said contacting step as compared to an identical method in which a nuclear receptor antagonist or inverse agonist is not added in said contacting step.

34. A method of identifying a compound that modulates the transcriptional activity of a nuclear receptor dimer comprising the steps:
a) contacting a first nuclear receptor subunit and an optional second nuclear receptor subunit different from said first nuclear receptor subunit, with;
i) a nucleic acid comprising a nuclear receptor response element able to bind both subunits of a nuclear receptor dimer comprising said first nuclear receptor subunit and said second nuclear receptor subunit, if present;
ii) a compound comprising a prospective ligand of said first or optional second nuclear receptor subunit, if present; and
iii) a nuclear receptor co-factor which will directly or indirectly bind either said first nuclear receptor subunit, or said second nuclear receptor subunit if present, in a ligand dependent manner,
wherein said nuclear receptor co-factor is selected from the group consisting of N-COR and SMRT; and
b) detecting the association or dissociation of said co-factor with said first or second nuclear receptor subunit in the presence of said compound when compared to performing step 1) in the absence of said compound, as an indication that said compound modulates the transcription regulatory activity of said nuclear receptor dimer.

35. The method of claim 34 wherein said at least one nuclear receptor subunit is selected from the group consisting of RAR, RXR, ER alpha, ER beta, VDR, PPAR, the thyroid receptor, FXR, LXR, the insect ecdysone receptor, the glucocorticoid receptor, the androgen receptor, the progestin receptor, the mineralcorticoid receptor and the CarB receptor.

36. The method of claim 35 wherein said at least one nuclear receptor subunit is selected from the group consisting of the glucocorticoid receptor, the androgen receptor, the progestin receptor, and the mineralcorticoid receptor.

37. The method of claim 35 wherein said at least one nuclear receptor subunit is selected from the group consisting of RAR, RXR, ER, VDR, PPAR, the thyroid receptor, FXR, LXR, and the insect ecdysone receptor.

38. The method of claim 35 wherein said detecting step comprises immobilizing a complex comprising said nuclear receptor dimer and identifying the presence or absence of said co-factor which co-immobilizes in said complex as compared to an identical method in which a nuclear receptor antagonist or inverse agonist is not added in said contacting step.

39. The method of claim 38 wherein said identifying step comprises permitting an antibody selective therefor to bind said co-factor and directly or indirectly detecting the presence of said antibody.

40. The method of claim 38 wherein said contacting and immobilization steps take place in a microtiter-type vessel.

41. The method of claim 39 wherein said contacting and immobilization steps take place in a microtiter-type vessel.

42. A method of identifying a coactivator-selective compound comprising the steps:
a) contacting a first nuclear receptor subunit and an optional second nuclear receptor subunit different from said first nuclear receptor subunit, with;
i) a nucleic acid comprising a nuclear receptor response element able to bind both subunits of a nuclear receptor dimer comprising said first nuclear receptor subunit and said second nuclear receptor subunit, if present;
ii) a compound comprising a prospective ligand of said first or optional second nuclear receptor subunit, if present; and
iii) first and second nuclear receptor coactivators which will directly or indirectly bind either said first nuclear receptor subunit, or said second nuclear receptor subunit if present, in a ligand dependent manner; and
b) detecting the association of said first coactivator and said second coactivator with said first or second nuclear receptor subunit in the presence of said compound when compared to performing step 1) in the absence of said compound,
where a different extent of association of said first coactivator as compared to said second coactivator indicates that said compound modulates the transcriptional activity of the nuclear hormone receptor by recruiting one coactivator in preference to another coactivator.

43. The method of claim 42 wherein said contacting step is performed in vitro.

44. The method of claim 43 wherein during said contacting step said first nuclear receptor subunit, optional second nuclear receptor subunit, and nuclear receptor co-factor are contained in a cell lysate.

45. The method of claim 44 wherein said cell lysate is prepared from cells transfected with at least one nucleic acid vector expressing said first and said optional second nuclear receptor subunit, if present.

46. The method of claim 43 wherein said first or second nuclear receptor coactivator is endogenously expressed by cells from which said cell lysate is made.

47. A method of identifying a coactivator-selective compound comprising the steps:
a) contacting a first nuclear receptor subunit and an optional second nuclear receptor subunit different from said first nuclear receptor subunit, with;
i) a nucleic acid comprising a nuclear receptor response element able to bind both subunits of a nuclear receptor dimer comprising said first nuclear receptor subunit and said second nuclear receptor subunit, if present, wherein said first nuclear receptor subunit is selected from the group consisting of RAR, RXR, ER alpha, ER beta, VDR, PPAR, the thyroid receptor, FXR, LXR, the insect ecdysone receptor, the glucocorticoid receptor, the androgen receptor, the progestin receptor, the mineralcorticoid receptor and the CarB receptor;
ii) a compound comprising a prospective ligand of said first or optional second nuclear receptor subunit, if present; and
iii) first and second nuclear receptor coactivators which will directly or indirectly bind either said first nuclear receptor subunit, or said second nuclear receptor subunit if present, in a ligand dependent manner; and
b) detecting the association of said first coactivator and said second coactivator with said first or second nuclear receptor subunit in the presence of said compound when compared to performing step 1) in the absence of said compound,
where a different extent of association of said first coactivator as compared to said second coactivator indicates that said compound modulates the transcriptional activity of the nuclear hormone receptor by recruiting one coactivator in preference to another coactivator.

48. The method of claim 47 wherein said first nuclear receptor subunit is selected from the group consisting of a glucocorticoid receptor subunit, an androgen receptor subunit, a progestin receptor subunit, and a mineralcorticoid receptor subunit, and said nuclear receptor dimer is a homodimer.

49. The method of claim 47 wherein said first nuclear receptor subunit is selected from the group consisting of an RAR subunit, an RXR subunit, an ER alpha subunit, an ER beta subunit, a VDR subunit, a PPAR subunit, a thyroid receptor subunit, an FXR subunit, an LXR subunit, and an insect ecdysone receptor subunit, and wherein said second nuclear receptor subunit comprises an RXR subunit.

50. The method of claim 49 wherein said first nuclear receptor subunit is selected from the group consisting of an RAR subunit and an RXR subunit, and wherein said second nuclear receptor subunit comprises an RXR subunit.

51. A method of identifying a coactivator-selective compound comprising the steps:
a) contacting a first nuclear receptor subunit and an optional second nuclear receptor subunit different from said first nuclear receptor subunit, with;
i) a nucleic acid comprising a nuclear receptor response element able to bind both subunits of a nuclear receptor dimer comprising said first nuclear receptor subunit and said second nuclear receptor subunit, if present;
ii) a compound comprising a prospective ligand of said first or optional second nuclear receptor subunit, if present; and
iii) first and second nuclear receptor coactivators which will directly or indirectly bind either said first nuclear receptor subunit, or said second nuclear receptor subunit if present, in a ligand dependent manner; and
b) detecting the association of said first coactivator and said second coactivator with said first or second nuclear receptor subunit in the presence of said compound when compared to performing step 1) in the absence of said compound,
wherein said first and second nuclear receptor coactivators each is independently selected from the group consisting of SRC-1, N-CoA2, TATA box binding protein (TBP), Creb binding protein (CBP) and ACTR, and
where a different extent of association of said first coactivator as compared to said second coactivator indicates that said compound modulates the transcriptional activity of the nuclear hormone receptor by recruiting one coactivator in preference to another coactivator.

52. The method of claim 51, wherein said first nuclear receptor coactivator is SRC-1.

53. The method of claim 52, wherein said first and second nuclear receptor coactivators are SRC-1 and ACTR.

54. The method of claim 1 wherein said detecting step comprises separating the dimer and any associated first and second nuclear receptor coactivators from other components present during the contacting step and detecting the presence or absence of said first and second nuclear receptor coactivators co-separating with said dimer.

55. The method of claim 54 wherein said separating step comprises selectively adsorbing the nuclear receptor dimer and any associated first and second nuclear receptor coactivators to an affinity reagent, and determining the presence or absence of first and second nuclear receptor coactivators co-adsorbing with said dimer.

56. The method of claim 55 wherein said affinity reagent comprises a first antibody selectively binding said dimer.


SEQUENCE LISTING (13 sequences)

SEQ ID NO: 1 - DNA, 17 bp, Homo Sapiens
  misc_feature (7)...(11): n=A,T,C or G
aggtcannnnnaggtca

SEQ ID NO: 2 - protein, 5 aa, Homo sapiens
EGCKG

SEQ ID NO: 3 - protein, 5 aa, Homo sapiens
GSCKV

SEQ ID NO: 4 - DNA, 21 bp, Artificial Sequence
  Hybrid RAR/glucocorticoid receptor response element
agggttcaccgaaagaacagt

SEQ ID NO: 5 - DNA, 27 bp, Homo sapiens
agctttcaggtcaccaggaggtcagaa

SEQ ID NO: 6 - protein, 14 aa, Homo sapiens
GKPIPNPLLGLDST

SEQ ID NO: 7 - DNA, 27 bp, Artificial Sequence
  Mutant glucocorticoid DR5 response element
agcttagagaacaccgaaagaacacta

SEQ ID NO: 8 - protein, 22 aa, Artificial Sequence
  Nuclear co-activator LXD-containing peptide
LESKGHKKLLQLLTCSSDDRGH

SEQ ID NO: 9 - protein, 22 aa, Artificial Sequence
  Nuclear co-activator LXD-containing peptide
LLQEKHRILHKLLQNGNSPAEV

SEQ ID NO: 10 - protein, 22 aa, Artificial Sequence
  Nuclear co-activator LXD-containing peptide
KKKENNALLRYLLDRDDPSDAL

SEQ ID NO: 11 - protein, 22 aa, Artificial Sequence
  Nuclear co-activator LXD-containing peptide
KYSQTSHKLVQLLTTTAEQQLR

SEQ ID NO: 12 - protein, 22 aa, Artificial Sequence
  Nuclear co-activator LXD-containing peptide
SLTERHKILHRLLQEGSPSDIT

SEQ ID NO: 13 - protein, 22 aa, Artificial Sequence
  Nuclear co-activator LXD-containing peptide
KESKDHQLLRYLLDKDEKDLRS